(19) United States
(12) Patent Application Publication
(10) Pub. No.: US 2026/0115137 A1
(43) Pub. Date: Apr. 30, 2026

(54) STABLE FORMULATIONS OF A MULTIVALENT VHH BASED CYTOTOXIC T LYMPHOCYTE ASSOCIATED ANTIGEN 4 (CTLA4) BINDER BINDING TO HUMAN CTLA4 AND METHODS OF USE THEREOF

(71) Applicant: Merck Sharp & Dohme LLC, Rahway, NJ (US)

(72) Inventors: Jainik P. Panchal, Cranbury, NJ (US); Mark A. McCoy, Basking Ridge, NJ (US); Smita Raghava, Mahwah, NJ (US); Geetha Thiagarajan, Westfield, NJ (US); Venus Hashemi, Manalapan, NJ (US)

(73) Assignee: Merck Sharp & Dohme LLC, Rahway, NJ (US)

(21) Appl. No.: 18/838,040

(22) PCT Filed: Feb. 22, 2023
(86) PCT No.: PCT/US2023/013584

§ 371 Date: Aug. 13, 2024

Related U.S. Application Data
(60) Provisional application No. 63/313,373, filed on Feb. 24, 2022.

(51) Int. Cl. A61K 9/08; A61K 47/12; A61K 47/18; A61K 47/20; A61K 47/22; A61K 47/26; C07K 16/28

(52) U.S. Cl. CPC A61K 9/08; A61K 47/12; A61K 47/18; A61K 47/20; A61K 47/22; A61K 47/26; C07K 16/2818; C07K 2317/35; C07K 2317/569; C07K 2317/94

(57) ABSTRACT

The present disclosure relates to stable formulations comprising amino acid sequences and polypeptides binding to cytotoxic T-lymphocyte associated antigen 4 (CTLA4). In another aspect, the present disclosure relates, in part, to formulations comprising improved heavy-chain immunoglobulin single variable domains (also referred to herein as “ISVs” or “ISVDs”, or “multivalent V_{HH} single domain antibodies”) binding to CTLA4, as well as to proteins, polypeptides and other constructs, compounds, molecules or chemical entities that comprise such ISVDs. Also provided are methods of treating various cancers and chronic infections with the formulations of the disclosure.

### CROSS-REFERENCE TO RELATED APPLICATIONS

This application is a National Stage Application of International Patent Application No. PCT/US2023/013584 filed Feb. 22, 2023, which claims the benefit of U.S. Ser. No. 63/313,373 filed Feb. 24, 2022, the contents of which are herein incorporated by reference in their entirety.

### REFERENCE TO SEQUENCE LISTING SUBMITTED ELECTRONICALLY

The contents of the electronic sequence listing (25380-SEQLIST-7FEB2023.xml: Size: 412 bytes; and Date of Creation: Feb. 7, 2023) are herein incorporated by reference in their entirety.

### FIELD OF THE INVENTION

This disclosure relates to formulations of therapeutic antibodies and their use in treating various disorders. In one aspect, this disclosure relates to formulations comprising amino acid sequences and polypeptides binding to cytotoxic T-lymphocyte associated antigen 4 (CTLA4). In another aspect, the present disclosure relates, in part, to formulations comprising improved multivalent protein derived from variable heavy-chain immunoglobulin domains (also referred to herein as “ISVs” or “ISVDs” or “multivalent V_{HH} based CTLA4 binder”) binding to CTLA4, as well as to proteins, polypeptides and other constructs, compounds, molecules or chemical entities that comprise such ISVDs. Also provided are methods of treating various cancers and chronic infections with the formulations described herein.

### BACKGROUND OF THE INVENTION

Abrogating immune regulatory molecules such as cytotoxic T lymphocyte antigen 4 (CTLA4) represents a new and promising strategy to induce tumor regression, stabilize disease, and prolong survival by manipulation of the immune system. An anti-CTLA4 (aCTLA4) antibody, ipilimumab, is currently being sold for indications including melanoma, renal cell carcinoma, colorectal cancer, hepatocellular carcinoma, non-small cell lung cancer, and malignant pleural mesothelioma. Evidence of tumor regression with prolonged time to progression has been seen in patients with melanoma who received CTLA4 antibodies and durable responses have been observed with ipilimumab in patients with melanoma, ovarian cancer, prostate cancer, and renal cell cancer.

Full T-cell activation requires two signals. The first is initiated by T-cell receptor binding to tumor-associated antigens presented by antigen presenting cells (APCs) via major histocompatibility complexes I and II. The second signal is generated when the principal costimulatory receptor on the T cell, CD28, binds to B7 ligand subtypes CD80 and CD86 on the APC. The resulting dual signaling induces changes including T-cell proliferation and cytokine release, triggering, and then amplifying the immune response. In response to T-cell activation, CTLA4 is upregulated and competes with CD28 for CD80 and CD86 binding on APCs but with significantly higher affinity, therefore downregulating—or deactivating—the T cell. CTLA-4, therefore, downregulates T-cell responses and APC function, resulting in a decreased immune response to tumor-associated antigens and immune tolerance.

One method by which to inhibit CTLA4-mediated downregulation is by interfering with its interaction with its ligands by binding it with a single domain antibody. The possibility exists that heavy chain only antibodies, originating in llamas, could cause an unwanted anti-drug immune response, e.g., by binding of the V_{HH} antibody by pre-existing antibodies in the patient's serum. Thus, novel methods by which to humanize V_{HH} antibodies so as to decrease or eliminate such a response are particularly valuable.

### SUMMARY OF THE INVENTION

In one aspect, the disclosure includes a formulation of a multivalent V_{HH} based CTLA4 binder, comprising (i) multispecific immunoglobulin single variable domain (ISVD) such as a V_{HH} that binds to human CTLA4 by contacting human CTLA4 at one or more of the following residues VRVTVL (SEQ ID NO: 8, amino acids 33-38 of SEQ ID NO: 22), ADSQVTEVC (SEQ ID NO: 9, amino acids 41-49 of SEQ ID NO: 22) and CKVELMYPPPYYLG (SEQ ID NO: 10, amino acids 93-106 of SEQ ID NO: 22), e.g., all three sites; (ii) a buffer, (iii) a non-reducing sugar; (iv) a non-ionic surfactant; and an antioxidant.

In one embodiment, the formulation further comprises a chelator.

In one embodiment, the multivalent V_{HH} based CTLA4 binder binds to human cytotoxic T lymphocyte associated antigen 4 (CTLA4). In a further embodiment, the multivalent V_{HH} based CTLA4 binder enhances T cell activation and cytokine production by blocking the interaction of human CTLA4 with its ligands, human CD80 (B7.1) and human CD86 (B7.2). In a further embodiment, the multivalent V_{HH} based CTLA4 binder is a trivalent protein comprised of three distinct modules, wherein each distinct module is a V_{HH}. The three distinct modules comprises two human CTLA4 binding modules and a human albumin-binding half-life extension module. The 3 modules are connected through 35GS linkers, which are 7 repeats of the amino acids Gly-Gly-Gly-Gly-Ser (GS). The molecule is composed of a single peptide chain, containing one intra-module disulfide bond per binding domain. The theoretical (average) molecular mass derived from the amino acid sequence is 42,309 Da. In a further embodiment, the multivalent V_{HH} based CTLA4 binder is a trivalent V_{HH} based CTLA4 binder. In a further embodiment, the trivalent V_{HH} based CTLA4 binder contains minor amounts of O glycosylation, primarily on the 35GS linkers, yielding additional species with molecular masses of 42,441 and 42,941 Da, depending on the attached O glycans.

In a further embodiment, the trivalent V_{HH} based CTLA4 binder is a humanized, sequence-optimized anti-human CTLA4 trivalent V_{HH} based CTLA4 binder, which blocks human and non-human primate CTLA4 binding to its ligands, CD80 and CD86.

In an embodiment, the formulation comprises (i) about 10 mg/m to about 200 mg/ml of trivalent V_{HH} based CTLA4 binder, (ii) about 5 mM to about 20 mM buffer; (iii) about 6% to about 16% weight/volume (w/v) non-reducing sugar; (iv) about 0.01% to about 0.10% non-ionic surfactant; and (v) about 1 mM to about 20 mM anti-oxidant.

In an embodiment, the formulation comprises (i) about 10 mg/mL to about 200 mg/ml of trivalent V_{HH} based CTLA4 binder; (ii) about 5 mM to about 20 mM buffer; (iii) about 6% to about 8% weight/volume (w/v) non-reducing sugar; (iv) about 0.01% to about 0.10% non-ionic surfactant; and (v) about 1 mM to about 20 mM anti-oxidant.

In another embodiment, the formulation further comprises an anti-PD-1 antibody, e.g., pembrolizumab or nivolumab. In another embodiment, the formulation further comprises a chelator. In one embodiment the chelator is present in amount of about 1 μM to about 50 μM. In yet another embodiment, the chelator is DTPA.

In one embodiment, the formulation has a pH between 4.5-6.5. In particular embodiments, the pH of the formulation is from about pH 5.0 to about pH 6.0. In a further embodiment, the pH of the formulation is from about pH 5.3 to about pH 5.8. In another embodiment, the pH is 5.3. In another embodiment, the pH is 5.4. In one embodiment, the pH is 5.5. In one embodiment, the pH is 5.6. In a further embodiment, the pH is 5.7. In an embodiment, the pH is 5.8.

In one embodiment of the formulation, the buffer is L-histidine buffer or sodium acetate buffer, the non-reducing sugar is sucrose, the non-ionic surfactant is polysorbate 80, and the anti-oxidant is methionine, or a pharmaceutically acceptable salt thereof. In one embodiment, the anti-oxidant is L-methionine. In another embodiment, the anti-oxidant is a pharmaceutically acceptable salt of L-methionine, such as, for example, methionine HCl.

In another embodiment, the formulation comprises (i) about 10 mg/mL to about 200 mg/mL of a trivalent V_{HH} based CTLA4 binder. (ii) about 5 mM to about 20 mM L-histidine or about 5 mM to about 20 mM of sodium acetate buffer; (iii) about 6% to about 16% w/v sucrose; (iv) about 0.01% to 0.10% w/v polysorbate 80; and (v) about 1 mM to about 20 mM L-methionine.

In another embodiment, the formulation comprises (i) about 10 mg/mL to about 200 mg/mL of a trivalent V_{HH} based CTLA4 binder, (ii) about 5 mM to about 20 mM L-histidine or about 5 mM to about 20 mM of sodium acetate buffer; (iii) about 6% to about 9% w/v sucrose; (iv) about 0.01% to 0.10% w/v polysorbate 80; and (v) about 1 mM to about 20 mM L-methionine.

In another embodiment, the formulation comprises (i) about 10 mg/mL to about 200 mg/mL of a trivalent V_{HH} based CTLA4 binder, (ii) about 5 mM to about 20 mM L-histidine or about 5 mM to about 20 mM of sodium acetate buffer; (iii) 8% w/v sucrose; (iv) about 0.02% w/v polysorbate 80; and (v) about 1 mM to about 20 mM L-methionine.

In another embodiment, the formulation further comprises an anti-PD-1 antibody, e.g., pembrolizumab or nivolumab. In an embodiment, the formulation further comprises a chelator. In one embodiment, the chelator is present in an amount of about 1 μM to about 50 μM. In one embodiment, the chelator is DTPA. In one embodiment the buffer is a L-histidine buffer. In one embodiment, the formulation comprises about 8 mM to about 12 mM L-histidine. In another embodiment, the buffer is about 5 mM to about 10 mM of L-methionine. In a further embodiment, the formulation comprises polysorbate 80 at a weight ratio of approximately 0.02% w/v. In another embodiment, the formulation comprises about 0.20 mg/mL polysorbate 80. In one embodiment, the trivalent V_{HH} based CTLA4 binder formulation comprises sucrose at a weight ratio of about 7% (w/v). In one embodiment, the trivalent V_{HH} based CTLA4 binder formulation comprises sucrose at a weight ratio of about 8% (w/v).

In embodiments of the formulation, the concentration of the trivalent V_{HH} based CTLA4 binder is from about 10 mg/mL to about 100 mg/mL. In another embodiment, the concentration of the trivalent V_{HH} based CTLA4 binder is about 10 mg/mL, 12.5 mg/mL, 15 mg/mL, 20 mg/mL, 25 mg/mL, 50) mg/mL, 75 mg/mL, or 100) mg/mL. In one embodiment, the concentration of the trivalent V_{HH} based CTLA4 binder is 25 mg/mL. In an additional embodiment, the concentration of the trivalent V_{HH} based CTLA4 binder is about 50 mg/mL. In another embodiment, the concentration of the trivalent V_{HH} based CTLA4 binder is about 75 mg/mL. In a further embodiment, the concentration of the trivalent V_{HH} based CTLA4 binder is 100 mg/mL.

In one aspect, provided is a formulation comprising about 25 mg/mL of a trivalent V_{HH} based CTLA4 binder, about 10 mM sodium acetate, about 8% w/v sucrose, about 0.02% w/v polysorbate 80, and about 10 mM L-methionine.

In one aspect, provided is a formulation comprising about 50 mg/ml of a trivalent V_{HH} based CTLA4 binder, about 10 mM sodium acetate, about 8% w/v sucrose, about 0.02% w/v polysorbate 80, and about 10 mM L-methionine.

In one aspect, provided is a formulation comprising about 75 mg/mL of a trivalent V_{HH} based CTLA4 binder, about 10 mM sodium acetate, about 8% w/v sucrose, about 0.02% w/v polysorbate 80, and about 10 mM L-methionine.

In one aspect, provided is a formulation comprising about 100 mg/mL of a trivalent V_{HH} based CTLA4 binder, about 10 mM sodium acetate, about 8% w/v sucrose, about 0.02% w/v polysorbate 80, and about 10 mM L-methionine.

In one aspect of any of the above formulations, the formulation has a pH of about 4.5 to 5.5. In one aspect of any of the above formulations, the formulation has a pH of about 4.8 to 5.2. In another aspect, the formulation has a pH of about 4.9 to about 5.1. In another aspect, the formulation has a pH of about 5.0.

In one aspect of any of the above formulations, the formulation comprises an anti-PD1 antibody or antigen binding fragment thereof. In one aspect, the anti-PD1 antibody is pembrolizumab. In another aspect, the anti-PD1 antibody is nivolumab.

In another aspect, the formulation may further comprise a chelator. In one embodiment, the chelator is DTPA. In one embodiment, the chelator is EDTA. In one aspect, the chelator is present in an amount from about 1 μM to about 50 μM. In one embodiment, the formulation comprises about 5 μM of the chelator. In one embodiment, the formulation comprises about 10 μM of the chelator. In one embodiment, the formulation comprises about 15 μM of the chelator. In one embodiment, the formulation comprises about 20 μM of the chelator. In one embodiment, the formulation comprises about 25 μM of the chelator. In one embodiment, the formulation comprises about 30 μM of the chelator. In one embodiment, the formulation comprises about 35 μM of the chelator. In one embodiment, the formulation comprises about 40 μM of the chelator. In one embodiment, the formulation comprises about 45 μM of the chelator. In one embodiment, the formulation comprises about 50 μM of the chelator. In one embodiment, the chelating agent is DTPA, which is present at any of the amounts stated above. In another embodiment, the chelating agent is EDTA which is present at any of the amounts stated above.

In one embodiment of the formulation, after 6 months at 5° C. the % monomer of the trivalent V_{HH} based CTLA4 binder is ≥90% purity as determined by size exclusion chromatography. In another embodiment of the formulation, after 6 months at 5° C. the % monomer of the trivalent V_{HH} based CTLA4 binder is ≥95% main peak as determined by size exclusion chromatography. In another embodiment of the formulation, after 6 months at 5° C. the % heavy chain of the trivalent V_{HH} based CTLA4 binder is ≥90% as determined by reduced CE-SDS. In another embodiment of the formulation, after 6 months at 5° C. the % heavy chain of the trivalent V_{HH} based CTLA4 binder is ≥95% as determined by reduced CE-SDS.

The present disclosure also provides a formulation comprising a trivalent V_{HH} based CTLA4 binder comprising one or more (e.g., 2) immunoglobulin single variable domains (ISVDs) that bind to human CTLA4 comprising: CDR1 that comprises the amino acid sequence FYGMG (SEQ ID NO: 2. (amino acids 6-10 of SEQ ID NO: 5) or GGTFSFYGMG (SEQ ID NO: 5); CDR2 that comprises the amino acid sequence DIRTSAGRTYYADSVKG (SEQ ID NO: 3) or DIRTSAGRTY (SEQ ID NO: 6. (amino acids 1-10 of SEQ ID NO: 3)); CDR3 that comprises the amino acid sequence EPSGISGWDY (SEQ ID NO: 4).

The present disclosure also provides a formulation comprising a trivalent V_{HH} based CTLA4 binder comprising one or more (e.g., 2) immunoglobulin single variable domains (ISVDs) that bind to human CTLA4 comprising: CDR1 that comprises the amino acid sequence FYGMG (SEQ ID NO: 2) or GGTFSFYGMG (SEQ ID NO: 5); CDR2 that comprises the amino acid sequence DIRTSAGRTYYADSVKG (SEQ ID NO: 3) or DIRTSAGRTY (SEQ ID NO: 6); CDR3 that comprises the amino acid sequence EPSGISGWDY (SEQ ID NO: 4); optionally, wherein the ISVD comprises a mutation at residues 11 and 89 (e.g., L11V and/or V89L, for example (E1D, L11V, A14P, Q45R, A74S, K83R, V89L, M96P, Q108L) wherein said residue numbers are Kabat residue numbers.

The present disclosure provides a formulation comprising a trivalent V_{HH} based CTLA4 binder comprising CDR1, CDR2 and CDR3 of an immunoglobulin comprising amino acid sequence set forth in SEQ ID NO: 1; and optionally including any number of additional mutations that are set forth herein or otherwise, e.g., up to 10 (e.g., 1, 2, 3, 4, 5, 6, 7, 8, 9 or 10) additional mutations (e.g., point mutations, substitutions, deletions, insertions).

The present disclosure comprises a formulation comprising a trivalent V_{HH} based CTLA4 binder which comprises an amino acid sequence having at least 85% (e.g., 85, 86, 87, 88, 89, 90, 91, 92, 93, 94, 95, 96, 97, 98, 99, 99.5, 99.9 or 100%) sequence identity with the amino acid sequence set forth in wherein the CTLA4 binder or ISVD comprises CDR1, CDR2 and CDR3 of an immunoglobulin comprising an amino acid sequence set forth in SEQ ID NO: 1 wherein said CTLA4 binder or ISVD comprises at least one mutation with respect to the amino acid sequence set forth in SEQ ID NO: 1. The present disclosure also comprises a formulation comprising a trivalent V_{HH} based CTLA4 binder comprising the amino acid sequence selected from the group consisting of SEQ ID NO: 22.

The present disclosure also comprises a formulation comprising a trivalent V_{HH} based CTLA4 binder comprising a CTLA4 binding moiety that binds to CTLA4 which is linked to one or more molecules that bind to an epitope that is not the epitope to which the CTLA4 binding moiety binds (e.g., PD1, CTLA4, LAG3, BTLA and/or CD27).

The present disclosure comprises a formulation comprising a trivalent V_{HH} based CTLA4 binder wherein the binder comprises: CTLA4 binder SEQ ID NO: 11 (wherein X is D), 35GS linker SEQ ID NO: 16, CTLA4 binder SEQ ID NO: 11 (wherein X is E), 35GS linker SEQ ID NO: 16, Human serum albumin binder SEQ ID NO: 17, and Alanine.

The present disclosure also provides a formulation in an injection device (e.g., hypodermic needle and syringe) or vessel that comprises the trivalent V_{HH} based CTLA4 binder optionally in association with a further therapeutic agent.

The present disclosure also provides a formulation comprising a polynucleotide encoding the trivalent V_{HH} based CTLA4 binder which is in a vector.

The present disclosure also provides a formulation wherein the method for making a trivalent V_{HH} based CTLA4 binder formulation comprises introducing a polynucleotide encoding the trivalent V_{HH} based CTLA4 binder into a host cell (e.g., a CHO cell or Pichia cell) and culturing the host cell in a medium under conditions favorable to expression of immunoglobulin from said polynucleotide and, optionally, purifying the immunoglobulin from said host cell and/or said medium. The present disclosure provides for any trivalent V_{HH} based CTLA4 binder produced by such a method in the said formulation.

The present disclosure also provides a formulation for preventing CTLA4 on a T-cell from binding to CD80 and/or CD86 on an antigen-presenting cell comprising contacting said CTLA4 with a trivalent V_{HH} based CTLA4 binder optionally in association with a further therapeutic agent. The present disclosure also provides a formulation wherein the formulation enhances an immune response in the body of a subject and comprises administering an effective amount of a trivalent V_{HH} based CTLA4 binder to the subject (e.g., mammal such as a human) optionally in association with a further therapeutic agent. The present disclosure also provides a formulation for treating or preventing cancer or an infectious disease in the body of a subject comprising administering an effective amount of a trivalent V_{HH} based CTLA4 binder optionally in association with a further therapeutic agent to the subject. In an embodiment of the disclosure, the cancer is metastatic cancer, a solid tumor, a hematologic cancer, leukemia, lymphoma, osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, carcinoid cancer or liver cancer, breast cancer or gastric cancer. In an embodiment of the disclosure, the infectious disease is a bacterial infection, a viral infection or a fungal infection. For example, in an embodiment of the disclosure, the subject is administered a formulation comprising a further therapeutic agent or a therapeutic procedure in association with the trivalent V_{HH} based CTLA4 binder.

The present disclosure provides a formulation comprising a trivalent V_{HH} based CTLA4 binder that binds to human CTLA4 by contacting human CTLA4 at one or more of the following residues: VRVTVL (SEQ ID NO: 8, amino acids 33-38 of SEQ ID NO: 22). ADSQVTEVC (SEQ ID NO: 9, amino acids 41-49 of SEQ ID NO: 22) and CKVELMYPPPYYLG SEQ ID NO: 10. (amino acids 93-106 of SEQ ID NO: 22); wherein the ISVD comprises a mutation at residues 11 (e.g., L11V) and 89 (e.g., V89L) wherein said residue numbers are Kabat residue numbers.

The present disclosure provides a formulation comprising a trivalent V_{HH} based CTLA4 binder comprising an immunoglobulin single variable domain (ISVD) that binds to human CTLA4 by contacting human CTLA4 at one or more of the following residues: VRVTVL (SEQ ID NO: 8, amino acids 33-38 of SEQ ID NO: 22). ADSQVTEVC (SEQ ID NO: 9, amino acids 41-49 of SEQ ID NO: 22) and CKVELMYPPPYYLG SEQ ID NO: 10, (amino acids 93-106 of SEQ ID NO: 22); wherein the ISVD comprises a mutation at residues 11 (e.g., L11V) and 89 (e.g., V89L) wherein said residue numbers are Kabat residue numbers.

The present disclosure also provides a formulation comprising a trivalent V_{HH} based CTLA4 binder comprising an immunoglobulin single variable domain (ISVD) that binds to CTLA4.

The present disclosure also provides a formulation comprising a trivalent V_{HH} based CTLA4 binder that cross-blocks a CTLA4 binder set forth herein from binding to CTLA4.

The present disclosure also provides an injection device or vessel that comprises a formulation comprising any trivalent V_{HH} based CTLA4 binder set forth herein (e.g., comprising the amino acid sequence set forth in SEQ ID NO: 13 or SEQ ID NO: 15) optionally in association with a further therapeutic agent.

The present disclosure also provides a formulation comprising a polynucleotide (e.g., DNA) encoding any trivalent V_{HH} based CTLA4 binder set forth herein (e.g., comprising the amino acid sequence set forth in SEQ ID NO: 13 or SEQ ID NO: 15); e.g., comprising the nucleotide sequence of SEQ ID NO: 12 or SEQ ID NO: 14; or a vector comprising such a polynucleotide; or a host cell comprising such a polynucleotide or vector.

The present disclosure also provides a formulation wherein the method for making the trivalent V_{HH} based CTLA4 binder set forth herein (e.g., comprising the amino acid sequence set forth in SEQ ID NO: 13 or SEQ ID NO: 15) comprising introducing a polynucleotide encoding the trivalent V_{HH} based CTLA4 binder into a host cell (e.g., a CHO cell or Pichia cell) and culturing the host cell in a medium under conditions favorable to expression of said trivalent V_{HH} based CTLA4 binder from said polynucleotide and, optionally, purifying the trivalent V_{HH} based CTLA4 binder from said host cell and/or said medium as well as any trivalent V_{HH} based CTLA4 binder produced by such a method.

The present disclosure also provides a formulation for preventing CTLA4 from binding to CD80 or CD86 (e.g., in the body of a subject) comprising contacting said CTLA4 with the trivalent V_{HH} based CTLA4 binder (e.g., comprising the amino acid sequence set forth in SEQ ID NO: 13 or SEQ ID NO: 15) optionally in association with a further therapeutic agent; as well as a method for enhancing an immune response in the body of a subject (e.g., a human) comprising administering an effective amount of the trivalent V_{HH} based CTLA4 binder (e.g., comprising the amino acid sequence set forth in SEQ ID NO: 13 or SEQ ID NO: 15) to the subject optionally in association with a further therapeutic agent (e.g., pembrolizumab). In addition, the present disclosure provides a formulation for treating or preventing cancer or an infectious disease in the body of a subject comprising administering an effective amount of trivalent V_{HH} based CTLA4 binder (e.g., comprising the amino acid sequence set forth in SEQ ID NO: 13 or SEQ ID NO: 15) optionally in association with a further therapeutic agent (e.g., pembrolizumab) to the subject.

In an embodiment of the disclosure, the cancer is metastatic cancer, a solid tumor, a hematologic cancer, leukemia, lymphoma, osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, carcinoid cancer or liver cancer, breast cancer or gastric cancer; or wherein the infectious disease is a bacterial infection, a viral infection or a fungal infection. In an embodiment of the disclosure, the subject is administered a further therapeutic agent (e.g., pembrolizumab) or a therapeutic procedure in association with the trivalent V_{HH} based CTLA4 binder.

In one embodiment, the formulation is contained in a glass vial. In another embodiment, the formulation is contained in an injection device. In another embodiment, the formulation is a liquid formulation. In certain embodiments, the formulation is stable at refrigerated temperature (2-8° C.) for at least 3 months, preferably 6 months, and more preferably 1 year, and even more preferably up to through 2 years.

In one aspect, the formulation is frozen to at least below −70° C. In another embodiment, the formulation is a reconstituted solution from a lyophilized formulation. In another embodiment, the formulation is a reconstituted solution from a lyophilized formulation with the addition of histidine.

In one aspect, provided are methods of treating chronic infection or cancer in a mammalian subject (e.g., a human) in need thereof comprising: administering an effective amount of the trivalent V_{HH} based CTLA4 binder formulation.

### BRIEF DESCRIPTION OF THE DRAWINGS

FIG. 1—A line graph showing V_{HH} thermal stress comparing percent sucrose with respect to change of percent in high molecular weight.

FIG. 2—A ¹H NMR spectrum of the trivalent V_{HH} based CTLA4 binder (comprising three distinct modules where two are human CTLA4 binding modules and a human albumin-binding half-life extension module).

FIG. 3A—Overlay of ¹H NMR spectra of trivalent V_{HH} based CTLA4 binder with 0, 4, 8, 16 and 20% sucrose.

FIG. 3B—Spectra of the trivalent V_{HH} based CTLA4 binder ¹H, ¹³C sfHMQC data with 0% (left) and 20% (right) sucrose.

FIG. 4—Protein-enhanced Diffussion-Ordered SpectrocopY (DOSY) spectrum which can be used to simultaneously measure the diffusion constants of individual molecules in mixtures.

FIG. 5A—A scatterplot showing sucrose dependent diffusion behavior of a trivalent V_{HH} based CTLA4 binder and acetate, showing the diffusion behavior of trivalent V_{HH} based CTLA4 binder (▪) compared to expected diffusion based on solution viscosity (◯). The hydrodynamic radius (Rh) of the trivalent V_{HH} based CTLA4 binder has been calculated from the diffusion constant.

FIG. 5B—Scatterplot showing sucrose dependent diffusion behavior of trivalent V_{HH} based CTLA4 binder and acetate. In particular, this shows the diffusion behavior of acetate alone (▴) and acetate in the presence of the trivalent V_{HH} based CTLA4 binder (▪) shows the reduction of acetate-protein interactions at higher sucrose concentrations.

FIG. 6A—Proton R2 relaxation data reveals changes in motion as a function of sucrose for methyl groups, trivalent protein, and amino acid linker amides. A plot of transverse relaxation rate (R2) versus percent sucrose showing ¹H R2 for different regions of the protein show distinct behavior in response to sucrose.

FIG. 6B—Proton R2 relaxation data reveals changes in motion as a function of sucrose for methyl groups, trivalent protein, and amino acid linker amides. A plot of R1R2 (s⁻²) and R2/R1 reveals anisotropic behavior for different regions of the trivalent V_{HH} based CTLA4 binder. R1 refers to the longitudinal relaxation rate. R1R2 is the relaxation rate product that is relatively insensitive to viscosity. R2/R1 is proportional to the protein size. Arrows show the direction of increasing sucrose concentration for each region. Dramatically different behavior is observed for the linker amides.

FIG. 7—Line graph of R2 where trivalent V_{HH} based CTLA4 binder methyl R2 shows changes in motions in response to sucrose. The difference in trivalent V_{HH} based CTLA4 binder methyl R2 compared to expected R2 purely due to viscosity or protein compaction.

FIG. 8A—A line graph showing water protein broadening for solutions of buffer (2), protein (1), protein and sucrose (3), and sucrose (4). The water R2 is <1 S⁻¹ in protein and buffer samples with no sucrose.

FIG. 8B—A scatterplot showing Water proton R2 values as a function of sucrose for buffer-sucrose samples (∘) and for buffer-sucrose-protein samples (⋅). Sucrose dependent changes in water R2 values are attributed to increased water-protein interactions.

FIG. 9—Schematic showing proposed mechanism for asparagine deamidation. The ‘+’ represents a scale of change where ‘+++’ is a higher range of change as compared to ‘+’.

FIG. 10A, 10B, 10C, 10D, 10E, 10F—Series of line graphs showing summary of charge variants during early research stability at different temperatures. FIGS. 10A, 10C, and 10E depict the percent area versus time in months for the main, basic variant and variants at 5° C., 25° C., and 40° C. respectively. FIGS. 10B, 10D, and 10F depict changes in percent peak over time in months for individual acidic peaks over 6 months at 5° C., 25° C., and 40° C. respectively.

FIG. 11A—A scatterplot showing sucrose dependent diffusion behavior of a trivalent VHH based CTLA4 binder, showing the protein diffusion behavior before (▪) and after (●).

FIG. 11B—Scatterplot showing sucrose dependent diffusion behavior of trivalent VHH based CTLA4 binder and acetate. In particular, the diffusion behavior of acetate alone (●) and acetate in the presence of the trivalent VHH based CTLA4 binder (▴) shows the reduction of acetate-protein interactions at higher sucrose concentrations.

FIG. 12—Line graph of R2 where trivalent V_{HH} based CTLA4 binder methyl R2 shows changes in motions in response to sucrose. The difference in trivalent V_{HH} based CTLA4 binder methyl R2 compared to expected R2 is purely due to viscosity or protein compaction.

### DETAILED DESCRIPTION OF THE INVENTION

In one aspect, this disclosure provides formulations comprising anti-CTLA4 antibodies and antigen-binding fragments thereof comprising methionine. In a further aspect, the formulation optionally comprises a chelating agent.

### I. Definitions and Abbreviations

As used throughout the specification and appended claims, the following abbreviations apply:
API active pharmaceutical ingredient
CDR complementarity determining region in the immunoglobulin variable regions, defined using the Kabat numbering system, unless otherwise indicated
CHO Chinese hamster ovary
CI confidence interval
CTLA4 cytotoxic T lymphocyte associated antigen 4
DTPA diethylenetriaminepentaacetic acid
EC50 concentration resulting in 50% efficacy or binding
ELISA enzyme-linked immunosorbant assay
FFPE formalin-fixed, paraffin-embedded
FR framework region
HRP horseradish peroxidase
HNSCC head and neck squamous cell carcinoma
IC50 concentration resulting in 50% inhibition
IgG immunoglobulin G
ICH International Conference of Harmonization
IHC immunohistochemistry or immunohistochemical
mAb monoclonal antibody
MES 2-(N-morpholino) ethanesulfonic acid
NCBI National Center for Biotechnology Information
NSCLC non-small cell lung cancer
PCR polymerase chain reaction
PD-1 programmed death 1 (a.k.a. programmed cell death-1 and programmed death receptor 1)
PD-L1 programmed cell death 1 ligand 1
PD-L2 programmed cell death 1 ligand 2
PS80 polysorbate 80
TNBC triple negative breast cancer
V_{H} immunoglobulin heavy chain variable region
V_{HH} Single variable domain on a heavy chain
VK immunoglobulin kappa light chain variable region
V_{L} immunoglobulin light chain variable region
v/v volume per volume
WFI water for injection
w/v weight per volume

So that the present disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the disclosure to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, inhibiting the growth or survival of tumor cells, eliminating or reducing the size of one or more cancerous lesions or tumors, decreasing the level of one or more tumor markers, ameliorating, reducing the severity or duration of the cancer, prolonging the survival of a patient relative to the expected survival in a similar untreated patient.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the disclosure is any of progression free survival (PFS), disease free survival (DFS) or overall survival (OS). PFS, also referred to as “Time to Tumor Progression” indicates the length of time during and after treatment that the cancer does not grow and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the present disclosure may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test). Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the disclosure, most preferably a human. In some embodiments, the patient is an adult patient. In other embodiments, the patient is a pediatric patient.

The term “antibody” refers to any form of antibody that exhibits the desired biological 20) activity.

The amino-terminal portion of each heavy chain includes a variable region of about 100 to 110 or more amino acids primarily responsible for antigen recognition. The variable regions of each heavy chain pair form the antibody binding site. The carboxy-terminal portion of the heavy chain may define a constant region primarily responsible for effector function. Furthermore, human heavy chains are typically classified as mu, delta, gamma alpha, or epsilon, and define the antibody's isotype as IgM, IgD, IgG, IgA, and IgE, respectively. See generally, Fundamental Immunology Ch. 7 (Paul. W., ed., 2nd ed. Raven Press. N.Y. (1989).

Typically, the variable domains of the heavy comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.: NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As is well-known in the art, there are multiple conventions to define and describe the CDRs of a VH or V_{HH} (also referred to as VHH) molecule, such as the Kabat definition (which is based on sequence variability and is the most commonly used) and the Chotia definition (which is based on the location of the structural loop regions). Reference is for example made to the website www.bioinf.org.uk/abs/. For the purposes of the present specification and claims, even though the CDRs according to Kabat may also be mentioned, the CDRs are most preferably defined on the basis of the Abm definition (which is based on Oxford Molecular's AbM antibody modelling software), as this is considered to be an optimal compromise between the Kabat and Chotia definitions. See Sequences of Proteins of Immunological Interest, Kabat, et al.; National Institutes of Health, Bethesda, Md.: 5th ed.: NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883; Chothia & Lesk (1987) J. Mol. Biol. 196:901-917; Elvin A. Kabat. Tai Te Wu, Carl Foeller, Harold M. Perry, Kay S. Gottesman (1991) Sequences of Proteins of Immunological Interest; Protein Sequence and Structure Analysis of Antibody Variable Domains. In: Antibody Engineering Lab Manual (Ed.: Duebel, S. and Kontermann, R., Springer-Verlag, Heidelberg). In an embodiment, CDR determination is according to Kabat, e.g., wherein FR1 of a V_{HH} comprises the amino acid residues at positions 1-30, CDR1 of a V_{HH} comprises the amino acid residues at positions 31-35, FR2 of a V_{HH} comprises the amino acids at positions 36-49, CDR2 of a V_{HH} comprises the amino acid residues at positions 50-65, FR3 of a V_{HH} comprises the amino acid residues at positions 66-94, CDR3 of a V_{HH} comprises the amino acid residues at positions 95-102, and FR4 of a V_{HH} comprises the amino acid residues at positions 103-113.

In an embodiment, CDRs are determined according to Kontermann and Dübel (Eds., Antibody Engineering, vol 2, Springer Verlag Heidelberg Berlin, Martin, Chapter 3, pp. 33-51, 2010).

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

The term “pharmaceutically effective amount” or “effective amount” means an amount whereby sufficient therapeutic composition or formulation is introduced to a patient to treat a diseased or condition. One skilled in the art recognizes that this level may vary according to the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of =0.1%, 0.5%, 1%, 2%, 3%, 4%, 5%, or 10%.

Therapeutic proteins are inherently unstable in solution and are formulated to achieve desirable solution behavior. This is true even for well-behaved, thoroughly characterized, highly “developable” mAb scaffolds: the developability of novel modalities is potentially much more uncertain. For traditional mAbs and novel scaffolds typical protein formulation is a complicated mixture containing the active protein, buffer, stabilizers (polyols, sugars, antioxidants), surfactants, and other excipients with the goal of minimizing physical and chemical instability and imparting an acceptable shelf-life to the drug product. Protein-excipient interactions are typically weak, non-specific and can involve multiple solution components. For example, arginine addition is reported to stabilize proteins against aggregation by binding to unfolded protein. Arginine can, however, potentially bind to charged protein patches or even to aromatic sidechains of folded proteins, perhaps even site-specifically. Similar behavior can be anticipated for other amino acids. Excipients can have multiple effects that may or may not be specific. PS80 is thought to indirectly affect protein stability as a surfactant and a direct role in protein stabilization by direct binding.

Sugars such as sucrose and trehalose are thought to provide stabilization due to preferential exclusion of protein in their presence driving protein in a more conformationally compact structure. They improve conformational stability but have limited impact on colloidal stability of the protein. There is extensive literature detailing the impact of sugars on protein stability during freeze-thaw: lyophilization as well as during stability in liquid or lyophilized state. The protection effect may or may lead to conformational change depending on the protein under investigation. The overall impact of excipient addition on protein structure has been probed in the past directly, using circular dichroism or indirectly, by monitoring changes in melting temperatures or specific volumes. However, the mechanistic understanding of protein-excipient interactions is still lacking. Moreover, the impact of protein structure on protein-excipient interaction is challenging to evaluate due to the lack of high-resolution analytical techniques that are sensitive to both protein and excipients. NMR spectroscopy is a powerful tool for this application; capable of providing detailed assessments of therapeutic protein structure, interactions, and solution behavior. NMR structural fingerprints capture information about protein solution structure and conformation as well as probe for site-specific interactions. Diffusion profiling and dynamics measurements are used to understand self-association, multimer assembly, aggregation and the impact of sequence and formulation on molecular motions. An emerging solution NMR application is to study details of therapeutic protein formulations. NMR spectroscopy is perhaps the only method that is capable of simultaneously assessing weak, multi-component interactions while providing structure and dynamics details. In all experiments the structural content for all mixture components is encoded in a simple 1H NMR; folded protein, buffer, sucrose and surfactants can be detected and differentiated. Interaction details can be arrayed and analyzed as diffusion and relaxation data.

aCTLA4 is equivalent to anti-CTLA4. CTLA4 is equivalent to CTLA-4.

The term “immunoglobulin single variable domain” (also referred to as “ISV” or ISVD”) is generally used to refer to immunoglobulin variable domains (which may be heavy chain or light chain domains, including V_{H}, V_{HH} or VL domains) that can form a functional antigen-binding site without interaction with another variable domain (e.g. without a V_{H}/VL interaction as is required between the V_{H} and VL domains of conventional 4-chain monoclonal antibody). Examples of ISVDs will be clear to the skilled person and for example include Nanobodies® (including a V_{HH}, a humanized V_{HH} and/or a camelized Virs such as camelized human VHS). IgNAR, domains, (single domain) antibodies (such as dAbs™) that are V_{H} domains or that are derived from a VI domain and (single domain) antibodies (such as dAbs™) that are VL domains or that are derived from a VL domain. ISVDs that are based on and/or derived from heavy chain variable domains (such as V_{H} or V_{HH} domains) are generally preferred. Most preferably, an ISVD will be a V_{HH}.

A Single domain antibody (sdAb) is an antibody fragment consisting of a single monomeric variable antibody domain of the heavy chain, a V_{HH}. A single-domain antibody, or V_{HH} antibody fragment, is isolated from camelid animals and is also known as a Nanobody™ (a registered trademark of Ablynx NV, and generally as defined in WO 2008/020079 or WO2009/138519). A V_{HH} antibody fragment corresponds to the variable region of a heavy chain of a camelid antibody. A V_{HH} antibody fragment is a peptide chain of about 110 amino acids long, comprising one variable domain (V_{H}) of a heavy-chain antibody, or of a common IgG.

A binder can be used to target and manipulate proteins and/or protein function. Such protein binders can be based on various scaffolds, such as nanobodies or multivalent V_{HH} antibody fragments. Protein binders are small, protein-based affinity reagents that can selectively recognize and bind to a target protein and are increasingly being used to study protein function in living cells. One class of peptide-based binders that currently exist are those that are based on or derived from immunoglobulins (antibodies and derivates, such as single-chain variable fragments (scFvs) and nanobodies). Binders can be V_{HH} antibody fragments.

The V_{HH} antibody molecule facilitates the construction of multivalent molecules, or multivalent binders by genetically linking using the same or different V_{HH} building blocks. A trivalent V_{HH} based CTLA4 binder contains a combination of three different single variable heavy chains domains which are able to bind different tumor antigens.

Fusion of two V_{HH} antibodies recognizing distinct cell surface proteins yields a bispecific binder or bispecific V_{HH} based binder. A multivalent V_{HH} domain antibody contains a combination of at least two different single variable heavy chains domains which are able to bind different tumor antigens. Additionally, fusion of three V_{HH}'s recognizing distinct cell surface proteins yields a trivalent binder or trivalent V_{HH} based binder. A multivalent V_{HH} based CTLA4 binder comprising a combination of three independent single variable heavy chains is a trivalent V_{HH} based CTLA4 binder. A multivalent V_{HH} based CTLA4 binder is also know as a multivalent V_{HH} (antibody), also abbreviated MV-V_{HH}. A trivalent V_{HH} based CTLA4 binder is also know as a trivalent V_{HH} (antibody).

A monovalent CTLA4 binder (e.g., ISVD such as a V_{HH} antibody molecule) is a molecule that comprises a single antigen-binding domain. A bivalent CTLA4 binder (e.g., ISVD such as a V_{HH} antibody molecule) comprises two antigen-binding domains. A trivalent CTLA4 binder (e.g., ISVD such as a VIII based binder) comprises three antigen-binding domains. A multivalent CTLA4 binder comprises more than one antigen-binding domain (e.g., 2, 3, 4, 5, 6, or 7).

A monospecific CTLA4 binder (e.g., ISVD such as a Vim antibody) binds a single antigen (CTLA4): a bispecific CTLA4 binder binds to two different antigens and a multispecific CTLA4 binder binds to more than one antigen.

A biparatopic CTLA4 binder (e.g., ISVD such as a V_{HH} antibody) is monospecific but binds to two different epitopes of the same antigen. A multiparatopic CTLA4 binder binds the same antigen to two or more epitope in the antigen.

A multispecific binder is a molecule that comprises a first CTLA4 binding moiety (e.g., a V_{HH} based binder, an ISVD, or a Vim antibody) and one or more (e.g., 1, 2, 3, 4, 5) additional binding moieties (e.g., V_{HH} antibody, an ISVDs,) that bind to an epitope other than that of the first CTLA4 binding moiety (e.g., a different epitope of CTLA4, or to CD27, LAG3, PD1 or BTLA).

A CTLA4 binder is an V_{HH} based CTLA4 binder, CTLA4 ISVD, or a CTLA4 Nanobody®. A CTLA4 single domain antibodies (sdAb) refers to a binder, ISVD, Nanobody®, or sdAb, respectively, that binds to CTLA4.

A binding moiety or binding domain or binding unit is a molecule, such as an V_{HH} binder, ISVD, or Nanobody®, that binds to an antigen such as CTLA4. A binding moiety or binding domain or binding unit may be part of a larger molecule such as a multivalent or multispecific binder that includes more than one moiety, domain or unit which may comprises another functional element, such as, for example, a half-life extender (HLE), targeting unit and/or a small molecule such a polyethyleneglycol (PEG).

Specifically, F023700912 (F912) and F023700914 (F914) are humanized, sequence-optimized anti-human Cytotoxic T Lymphocyte Associated Antigen 4 (CTLA4) Nanobodies®, or trivalent V_{HH} based CTLA4 binders, which block human and nonhuman primate CTLA4 binding to its ligands, CD80 and CD86. F023700912 (F912) is comprised of three distinct modules. F912 has two CTLA4 domains fused to albumin-binding domain to form trivalent V_{HH} based CTLA4 binder. F912 is also referred to as a aCTLA4-aCTLA4 V_{HH} based binder or Nanobody®. F023700912 (F912) and F023700914 (F914) are examples of V_{HH} based CTLA4 binder and can also be referred to as multispecific binders.

The term “half-life” as used herein in relation to a CTLA4 binder (e.g., ISVD such as a Nanobody) or other molecule can generally be defined as described on page 57 of WO 2008/020079 and as mentioned therein refers to the time taken for the serum concentration of the amino acid sequence, CTLA4 binder, compound or polypeptide to be reduced by 50%, in vivo, for example due to degradation of the sequence or compound and/or clearance or sequestration of the sequence or compound by natural mechanisms. The in vivo half-life of an amino acid sequence, CTLA4 binder, compound or polypeptide of the invention can be determined in any manner known, such as by pharmacokinetic analysis. Suitable techniques will be clear to the person skilled in the art and may, for example, generally be as described on page 57 of WO 2008/020079. As also mentioned on page 57 of WO 2008/020079, the half-life can be expressed using parameters such as the t½-alpha, t½-beta and the area under the curve (AUC). In this respect it should be noted that the term “half-life” as used herein in particular refers to the t½-beta or terminal half-life (in which the t½-alpha and/or the AUC or both may be kept out of considerations). Reference is for example made to the Experimental Part below, as well as to the standard handbooks, such as Kenneth, A et al.: Chemical Stability of Pharmaceuticals: A Handbook for Pharmacists and Peters et al., Pharmacokinete analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined on page 57 of WO 2008/020079 and in particular refer to an increase in the t½-beta, either with or without an increase in the t½-alpha and/or the AUC or both.

“Human serum albumin binders” or “HSA binders” of the present invention are any of the molecules described herein that bind to HSA (e.g., an ISVD such as a Nanobody) as well as any antibody or antigen-binding fragment thereof that binds to HSA and includes any of the HSA binding moieties described herein. An individual HSA binder may be referred to has a HSA binding moiety if it is part of a larger molecule, e.g., a multivalent molecule such as F023700912 or F023700914

As used herein, “x % (w/v)” is equivalent to x g/100 mL (for example 5% w/v equals 50) mg/mL).

Formulations of the present disclosure include antibodies and CTLA-4 binders thereof that are biologically active when reconstituted or in liquid form.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma. Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Chothia” means an antibody numbering system described in Al-Lazikani et al., JMB 273:927-948 (1997).

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin.

“Hypervariable region” refers to the amino acid residues of an antibody that are responsible for antigen-binding. The hypervariable region comprises amino acid residues from a “complementarity determining region” or “CDR” (e.g. residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.).

As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

The amino acid residues of a trivalent V_{HH} based CTLA4 binder are numbered according to the general numbering for VHs given by Kabat et al. (“Sequence of proteins of immunological interest”, US Public Health Services, NIH Bethesda, MD, Publication No. 91), as applied to V_{HH} domains from Camelids in the article of Riechmann and Muyldermans, J. Immunol. Methods 2000 Jun. 23:240 (1-2): 185-195; or referred to herein. It should be noted that, as is well known in the art for V_{H} domains and for V_{HH} domains, the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of V_{H} domains, which methods can also be applied in an analogous manner to V_{HH} domains from Camelids and trivalent V_{HH} based CTLA4 binder, are the method described by Chothia et al. (Nature 342, 877-883 (1989)), the so-called “AbM definition” and the so-called “contact definition”. However, in the present description, aspects and figures, the numbering according to Kabat as applied to V_{HH} domains by Riechmann and Muyldermans will be followed, unless indicated otherwise.

“Conservatively modified variants” or “conservative substitution” refers to substitutions of amino acids are known to those of skill in this art and may be made generally without altering the biological activity of the resulting molecule, even in essential regions of the polypeptide. Such exemplary substitutions are preferably made in accordance with those set forth in Table A.

TABLE A
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See. e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the disclosure, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g., by measuring the dimensions of tumor(s) upon removal from the subject. e.g., using calipers, or while in the body using imaging techniques, e.g., bone scan, ultrasound, CT or MRI scans.

The term “buffer” encompasses those agents which maintain the solution pH of the formulations of the disclosure in an acceptable range, or, for Lyophilized formulations of the disclosure, provide an acceptable solution pH prior to lyophilization.

The terms “lyophilization.” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g., parenteral administration), and may optionally be suitable for subcutaneous administration. “Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e., % high molecular weight species as determined, for example, by HP-SEC.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

### II. Formulations of the Present Disclosure

In one aspect, the disclosure provides stable biological formulations comprising a trivalent V_{HH} based CTLA4 binder which specifically binds to human CTLA4 as the active pharmaceutical ingredient.

### Trivalent V_{HH} Based CTLA4 Binder

The present disclosure aims to provide formulations comprising a trivalent V_{HH} based CTLA4 binder, in particular improved CTLA4 ISVDs and more in particular improved CTLA4 single domain antibodies. A trivalent V_{HH} based CTLA4 binder of the present disclosure includes polypeptides which are variants of polypeptides comprising the amino acid sequence of SEQ ID NO: 1.

The scope of the present disclosure includes a formulation comprising a trivalent V_{HH} based CTLA4 binder set forth herein (e.g., F023700912) that bind to the same CTLA4 epitope of such binders. Additionally, WO2008071447 and WO2017087588 describe trivalent V_{HH} based CTLA4 binder's that can bind to CTLA4 and uses thereof.

TABLE 1
V_{HH} based CTLA4 binders F023700912/F023700914

| | | |
|---|---|---|
| SEQ ID NO: 1 | F023700912 | DVQLVESGGGVVQPGGSLRLSCAASGGTFSFYGMG |
| | F023700914 | WFRQAPGKEREFVADIRTSAGRTYYADSVKGRFTISR |
| | | DNSKNTVYLQMNSLRPEDTALYYCAAEPSGISGWDY |
| | | WGQGTLVTVSS |
| SEQ ID NO: 2 | CDR1 (Kabat) | FYGMG |
| | | (amino acids 6-10 of SEQ ID NO: 5) |
| SEQ ID NO: 3 | CDR2 (Kabat) | DIRTSAGRTYYADSVKG |
| SEQ ID NO: 4 | CDR3 | EPSGISGWDY |
| | (Kabat/Abm) | |
| SEQ ID NO: 5 | CDR1 (Abm) | GGTFSFYGMG |
| SEQ ID NO: 6 | CDR2 (Abm) | DIRTSAGRTY |
| | | (amino acids 1-10 of SEQ ID NO: 3) |
| SEQ ID NO: 7 | CDR3 | EPSGISGWDY |
| | (Kabat/Abm) | |

*CDRs underscored and/or bold
Note:
SEQ ID NO: 4 is identical to SEQ ID NO: 7

The present disclosure includes any trivalent V_{HH} based CTLA4 binder comprising the amino acid sequence of SEQ ID NO: 1 or an amino acid sequence comprising 80% or more (e.g., 85%, 90%, 95%, 96%, 97%, 98% or 99%) amino acid sequence identity wherein the trivalent V_{HH} based CTLA4 binder retains the ability to bind to CTLA4.

As mentioned, the trivalent V_{HH} based CTLA4 binder described herein can bind (and in particular, can specifically bind) to CTLA4. In an embodiment of the disclosure, the trivalent V_{HH} based CTLA4 binder can bind to CTLA4 and thereby prevent CD80, and CD86 on antigen-presenting cells from binding to CTLA4 on T cells. In an embodiment of the disclosure, the resulting blockade of CTLA4 signaling prolongs T-cell activation, restores T-cell proliferation, and thus amplifies T-cell-mediated immunity, which theoretically enhances the patient's capacity to mount an antitumor immune response.

In an embodiment of the disclosure, a trivalent V_{HH} based CTLA4 binder, has one or more of the following properties:
Binds to CTLA4 (e.g., human and/or cynomolgous monkey CTLA4 (CTLA4-Fc fusion protein), e.g., with a K_{D} of about 1 nM (e.g., 1.2 nM);
Binds to CTLA4 on the surface of a cell, e.g., a CHO cell expressing CTLA4:
Blocks binding of CD80 or CD86 to CTLA4 (e.g., CTLA4 expressed on CHO cells);
Does not bind to BTLA, CD8, PD1, LAG3, and/or CD28:
Binds in vitro and/or in vivo to human, rhesus monkey and mouse serum albumin (when fused to one or more ALB11002 binders):
Inhibits tumor growth (e.g., of pancreatic tumors, e.g., human pancreatic tumors in a mouse harboring human immune cells)

A trivalent V_{HH} based CTLA4 binder preferably comprise the following CDRs (according to the Kabat convention):
a CDR1 (according to Kabat) that comprises the amino acid sequence FYGMG (SEQ ID NO: 2); and
a CDR2 (according to Kabat) that comprises the amino acid sequence DIRTSAGRTYYADSVKG (SEQ ID NO: 3); and
a CDR3 (according to Kabat) that comprises the amino acid sequence EPSGISGWDY (SEQ ID NO: 4); optionally, wherein CDR1, CDR2 and/or CDR3 comprises 1, 2, 3, 4, 5, 5, 6, 7, 8, 9 or 10 substitutions, e.g., conservative substitutions.

Alternatively, when the CDRs are given according to the Abm convention, the trivalent V_{HH} based CTLA4 binder preferably comprise the following CDRs:
a CDR1 (according to Abm) that is the amino acid sequence GGTFSFYGMG (SEQ ID NO: 5); and
a CDR2 (according to Abm) that is the amino acid sequence DIRTSAGRTY (SEQ ID NO: 6); and
a CDR3 (according to Abm) that is the amino acid sequence EPSGISGWDY (SEQ ID NO: 7, which is the same as SEQ ID NO: 4); optionally, wherein CDR1, CDR2 and/or CDR3 comprises 1, 2, 3, 4, 5, 5, 6, 7, 8, 9 or 10 substitutions, e.g., conservative substitutions.

The present disclosure provides a trivalent V_{HH} based CTLA4 binder, F023700912, comprising amino acid sequence:

DVQLVESGGGVVQPGGSLRLSCAASGGTFSFYGMGWFRQAPGKEREFVADIRTSAGR
TYYADSVKGRFTISRDNSKNTVYLQMNSLRPEDTALYYCAAEPSGISGWDYWGQGTLV

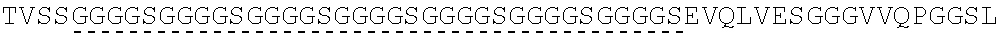

RLSCAASGGTFSFYGMGWFRQAPGKEREFVADIRTSAGRTYYADSVKGRFTISRDNSK

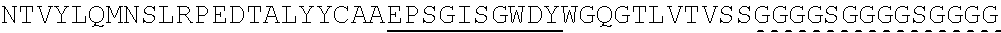

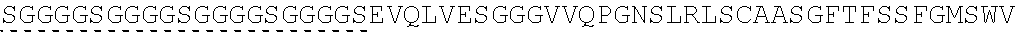

RQAPGKGLEWVSSISGSGSDTLYADSVKGRFTISRDNAKTTLYLQMNSLRPEDTALYYC
TIGGSLSRSSQGTLVTVSSA

(SEQ ID NO: 13; 35GS linkers underscored with dotted line; CDRs underscored and/or bold). Optionally, the first residue of any binder moiety in the molecule is substituted with a D or an E as appropriate.

Optionally, the trivalent VEH based CTLA4 binder comprises a signal peptide such as:

(SEQ ID NO: 21)
MRFPSIFTAVLFAASSALAAPVNTTTEDETAQIPAEAVIGYSDLEGDF
DVAVLPFSNSTNNGLLFINTTIASIAAKEEGVSLEKR

F023700912 can be encoded by a polynucleotide comprising the following nucleotides:

gacgtgcaat tggtggagtc tgggggagga gtggtgcagc cggggggctc tctgagactc 60
tcctgtgcag cctctggtgg caccttcagt ttctatggca tgggctggtt ccgccaggct 120
ccagggaagg agcgcgagtt tgtagcagat attagaacca gtgctggtag gacatactat 180
gcagactccg tgaagggccg attcaccatc tccagagaca acagcaagaa cacggtgtat 240
ctgcaaatga acagcctgcg ccctgaggac acggccctgt attactgtgc agcagagcca 300
agtggaataa gtggttggga ctactggggc caggggaccc tggtcacggt ctcctccgga 360
ggcggtgggt caggtggcgg aggcagcggt ggaggaggta gtggcggtgg cggtagtggg 420
ggtggaggca gcggaggcgg aggcagtggg ggcggtggat ccgaggtgca gttggtggag 480
tctgggggag gagtggtgca gccggggggc tctctgagac tctcctgtgc agcctctggt 540
ggcaccttca gtttctatgg catgggctgg ttccgccagg ctccagggaa ggagcgcgag 600
tttgtagcag atattagaac cagtgctggt aggacatact atgcagactc cgtgaagggc 660
cgattcacca tctccagaga caacagcaag aacacggtgt atctgcaaat gaacagcctg 720
cgccctgagg acacggccct gtattactgt gcagcagagc caagtggaat aagtggttgg 780
gactactggg gccaggggac cctggtcacg gtctcgagcg gaggcggtgg gtcaggtggc 840
ggaggcagcg gtggaggagg tagtggcggt ggcggtagtg ggggtggagg cagcggaggc 900
ggaggcagtg ggggcggtgg ctcagaggta caactagtgg agtctggagg tggcgttgtg 960
caaccgggta acagtctgcg ccttagctgc gcagcgtctg gctttacctt cagctccttt 1020
ggcatgagct gggttcgcca ggctccggga aaaggactgg aatgggtttc gtctattagc 1080
ggcagtggta gcgatacgct ctacgcggac tccgtgaagg gccgtttcac catctcccgc 1140
gataacgcca aaactacact gtatctgcaa atgaatagcc tgcgtcctga agatacggcc 1200
ctgtattact gtactattgg tggctcgtta agccgttctt cacagggtac cctggtcacc 1260
gtctcctcag cg 1272

(SEQ ID NO: 12; optionally lacking the signal sequence of nucleotides 1-255)

F023700912 comprises the following moieties:
CTLA4 binder 11F01(E1D,L11V,A14P,Q45R,A74S,K83R, V89L,M96P,Q108L)
35 GS linker
CTLA4 binder 11F01(L11V,A14P,Q45R,A74S,K83R, V89L,M96P,Q108L)
35 GS linker
Human serum albumin binder ALB11002;
C-terminal extension alanine.

Specifically, F023700912 comprises:
CTLA4 binder SEQ ID NO: 11
35GS linker SEQ ID NO: 16
CTLA4 binder SEQ ID NO: 11 (wherein X is D or E)
35GS linker SEQ ID NO: 16
Human serum albumin binder SEQ ID NO: 17
Alanine
   (the present disclosure includes any binder including such an arrangement of moieties)

The present disclosure also provides a V_{HH} based CTLA4 binder, F023700914, comprising amino acid sequence:

DVQLVESGGGVVQPGGSLRLSCAASGGTFSFYGMGWFRQAPGKEREFVADIRTSAGR
TYYADSVKGRFTISRDNSKNTVYLQMNSLRPEDTALYYCAAEPSGISGWDYWGQGTLV

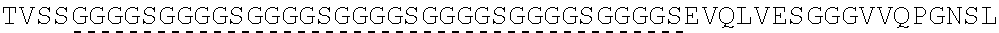

RLSCAASGFTFSSFGMSWVRQAPGKGLEWVSSISGSGSDTLYADSVKGRFTISRDNAKT
TLYLQMNSLRPEDTALYYCTIGGSLSRSSQGTLVTVSSA

(SEQ ID NO: 15; 35GS linker underscored with dotted line; CDRs underscored and/or bold). Optionally, the first residue of any binder moiety in the molecule is substituted with a D or an E as appropriate.

Optionally, the V_{HH} based CTLA4 binder comprises a signal peptide such as:

(SEQ ID NO: 21)
MRFPSIFTAVLFAASSALAAPVNTTTEDETAQIPAEAVIGYSDLEGDF
DVAVLPFSNSTNNGLLFINTTIASIAAKEEGVSLEKR

F023700914 can be encoded by a polynucleotide comprising the following nucleotides:

gacgtgcaat tggtggagtc tgggggagga gtggtgcagc cggggggctc tctgagactc 60
tcctgtgcag cctctggtgg caccttcagt ttctatggca tgggctggtt ccgccaggct 120
ccagggaagg agcgcgagtt tgtagcagat attagaacca gtgctggtag gacatactat 180
gcagactccg tgaagggccg attcaccatc tccagagaca acagcaagaa cacggtgtat 240
ctgcaaatga acagcctgcg ccctgaggac acggccctgt attactgtgc agcagagcca 300
agtggaataa gtggttggga ctactggggc caggggaccc tggtcacggt ctcctccgga 360
ggcggtgggt caggtggcgg aggcagcggt ggaggaggta gtggcggtgg cggtagtggg 420
ggtggaggca gcggaggcgg aggcagtggg ggcggtggat ccgaggtgca gttggtggag 480
tctggaggtg gcgttgtgca accgggtaac agtctgcgcc ttagctgcgc agcgtctggc 540
tttaccttca gctcctttgg catgagctgg gttcgccagg ctccgggaaa aggactggaa 600
tgggtttcgt ctattagcgg cagtggtagc gatacgctct acgcggactc cgtgaagggc 660
cgtttcacca tctcccgcga taacgccaaa actacactgt atctgcaaat gaatagcctg 720
cgtcctgaag atacggccct gtattactgt actattggtg gctcgttaag ccgttcttca 780
cagggtaccc tggtcaccgt ctcctcagcg 810

(SEQ ID NO: 14; optionally lacking the signal sequence of nucleotides 1-255)

F023700914 comprises the following moieties:
CTLA4 binder 11F01 (E1D,L11V,A14P,Q45R,A74S,K83R,V89L,M96P,Q108L);
35 GS linker;
Human serum albumin binder ALB11002;
C-terminal extension alanine.

Specifically, F023700914 comprises:
CTLA4 binder SEQ ID NO: 11 wherein D is X or E
35GS linker SEQ ID NO: 16
Human serum albumin binder SEQ ID NO: 17
Alanine
   (the present disclosure includes any binder including such an arrangement of moieties)

The present disclosure includes any multispecific CTLA4 binder comprising the amino acid sequence of SEQ ID NO: 13 or 15 or an amino acid sequence comprising 80% or more (e.g., 85%, 90%, 95%, 96%, 97%, 98% or 99%) amino acid sequence identity wherein the CTLA4 binder retains the ability to bind to CTLA4 and, optionally, HSA.

TABLE 2
V_{HH} based CTLA4 binder sequences summary table including Human Serum
Albumin (HSA) Binder ALB11002

| | SEQ |
|---|---|
| | ID |
| SEQUENCE | NO: |
| VRVTVL | 8 |
| ADSQVTEVC | 9 |
| CKVELMYPPPYYLG | 10 |
| XVQLVESGGGVVQPGGSLRLSCAASGGTFSFYGMGWFRQAPGKERE | 11 |
| FVADIRTSAGRTYYADSVKGRFTISR | |
| DNSKNTVYLQMNSLRPEDTALYYCAAEPSGISGWDYWGQGTLVTVS | |
| S | |
| Wherein X is D or E. | |
| (Amino acid sequence of ISVD/CTLA4 Binder) | |
| gacgtgcaat tggtggagtc tgggggagga gtggtgcagc cggggggctc tctgagactc 60 | 12 |
| tcctgtgcag cctctggtgg caccttcagt ttctatggca tgggctggtt ccgccaggct 120 | |
| ccagggaagg agcgcgagtt tgtagcagat attagaacca gtgctggtag gacatactat 180 | |
| gcagactccg tgaagggccg attcaccatc tccagagaca acagcaagaa cacggtgtat 240 | |
| ctgcaaatga acagcctgcg ccctgaggac acggccctgt attactgtgc agcagagcca 300 | |
| agtggaataa gtggttggga ctactggggc caggggaccc tggtcacggt ctcctccgga 360 | |
| ggcggtgggt caggtggcgg aggcagcggt ggaggaggta gtggcggtgg cggtagtggg 420 | |
| ggtggaggca gcggaggcgg aggcagtggg ggcggtggat ccgaggtgca gttggtggag 480 | |
| tctgggggag gagtggtgca gccggggggc tctctgagac tctcctgtgc agcctctggt 540 | |
| ggcaccttca gtttctatgg catgggctgg ttccgccagg ctccagggaa ggagcgcgag 600 | |
| tttgtagcag atattagaac cagtgctggt aggacatact atgcagactc cgtgaagggc 660 | |
| cgattcacca tctccagaga caacagcaag aacacggtgt atctgcaaat gaacagcctg 720 | |
| cgccctgagg acacggccct gtattactgt gcagcagagc caagtggaat aagtggttgg 780 | |
| gactactggg gccaggggac cctggtcacg gtctcgagcg gaggcggtgg gtcaggtggc 840 | |
| ggaggcagcg gtggaggagg tagtggcggt ggcggtagtg ggggtggagg cagcggaggc | |
| 900 | |
| ggaggcagtg ggggcggtgg ctcagaggta caactagtgg agtctggagg tggcgttgtg 960 | |
| caaccgggta acagtctgcg ccttagctgc gcagcgtctg getttacctt cagctccttt 1020 | |
| ggcatgagct gggttcgcca ggctccggga aaaggactgg aatgggtttc gtctattagc 1080 | |
| ggcagtggta gcgatacgct ctacgcggac tccgtgaagg gccgtttcac catctcccgc 1140 | |
| gataacgcca aaactacact gtatctgcaa atgaatagcc tgcgtcctga agatacggcc 1200 | |
| ctgtattact gtactattgg tggctcgtta agccgttctt cacagggtac cctggtcacc 1260 | |
| gtctcctcag cg | |
| (nucleotide sequence of trivalent V_{HH} based CTLA4 binder - F023700912) | |
| DVQLVESGGGVVQPGGSLRLSCAASGGTFSFYGMG | 13 |
| WFRQAPGKEREFVADIRTSAGRTYYADSVKGRFTISRDNSKNTVYL | |
| QMNSLRPEDTALYY | |
| 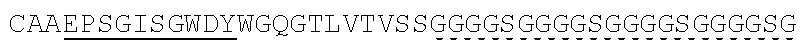 | |
| 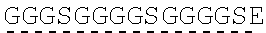 | |
| VQLVESGGGVVQPGGSLRLSCAASGGTFSFYGMGWFRQAPGKERE | |
| FVADIRTSAGRTYYA | |
| DSVKGRFTISRDNSKNTVYLQMNSLRPEDTALYYCAAEPSGISGWDY | |
| WGQGTLVTVSSGG | |
| 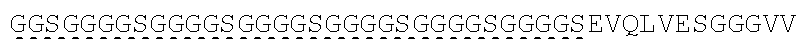 | |
| QPGNSLRLSCAASGF | |
| TFSSFGMSWVRQAPGKGLEWVSSISGSGSDTLYADSVKGRFTISRDN | |
| AKTTLYLQMNSLR | |
| PEDTALYYCTIGGSLSRSSQGTLVTVSSA | |
| (amino acid sequence of trivalent V_{HH} based CTLA4 binder - F023700912) | |
| gacgtgcaat tggtggagtc tgggggagga gtggtgcagc cggggggctc tctgagactc 60 | 14 |
| tcctgtgcag cctctggtgg caccttcagt ttctatggca tgggctggtt ccgccaggct 120 | |
| ccagggaagg agcgcgagtt tgtagcagat attagaacca gtgctggtag gacatactat 180 | |
| gcagactccg tgaagggccg attcaccate tocagagaca acagcaagaa cacggtgtat 240 | |
| ctgcaaatga acagcctgcg ccctgaggac acggccctgt attactgtgc agcagagcca 300 | |
| agtggaataa gtggttggga ctactggggc caggggaccc tggtcacggt ctcctccgga 360 | |
| ggcggtgggt caggtggcgg aggcagcggt ggaggaggta gtggcggtgg cggtagtggg 420 | |
| ggtggaggca gcggaggcgg aggcagtggg ggcggtggat ccgaggtgca gttggtggag 480 | |
| tctggaggtg gcgttgtgca accgggtaac agtctgcgcc ttagctgcgc agcgtctggc 540 | |
| tttaccttca gctcctttgg catgagctgg gttcgccagg ctccgggaaa aggactggaa 600 | |
| tgggtttcgt ctattagegg cagtggtagc gatacgctct acgeggactc cgtgaagggc 660 | |
| cgtttcacca tctcccgega taacgccaaa actacactgt atctgcaaat gaatagcctg 720 | |
| cgtcctgaag atacggccct gtattactgt actattggtg gctcgttaag ccgttcttca 780 | |
| cagggtaccc tggtcaccgt ctcctcagcg | |
| (nucleotide sequence of V_{HH} based CTLA4 binder - F023700914) | |
| DVQLVESGGGVVQPGGSLRLSCAASGGTFSFYGMG | 15 |
| WFRQAPGKEREFVADIRTSAGRTYYADSVKGRFTISRDNSKNTVYL | |
| QMNSLRPEDTALYY | |
| 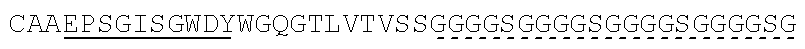 | |
| 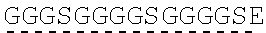 | |
| VQLVESGGGVVQPGNSLRLSCAASGFTFSSFGMSWVRQAPGKGLEW | |
| VSSISGSGSDTLYA | |
| DSVKGRFTISRDNAKTTLYLQMNSLRPEDTALYYCTIGGSLSRSSQGT | |
| LVTVSSA | |
| (amino acid sequence of V_{HH} based CTLA4 binder - F023700914) | |
| GGGGSGGGGSGGGGSGGGGSGGGGSGGGGSGGGGS | 16 |
| (35GS Linker) | |
| EVQLVESGGG XVQPGNSLRL SCAASGFTFS SFGMSWVRQA | 17 |
| PGKGLEWVSS ISGSGSDTLY | |
| ADSVKGRFTI SRDNAKTTLY LQMNSLRPED TAXYYCTIGG | |
| SLSRSSQGTL VTVSSA | |
| wherein X at residues 11 and 93 are L or V; | |
| for example, | |
| EVQLVESGGGVVQPGNSLRLSCAASGF | |
| TFSSFGMSWVRQAPGKGLEWVSSISGSGS | |
| DTLYADSVKGRFTISRDNAKTTLYLQMNSLR | |
| PEDTALYYCTIGGSLSRSSQGTLVTVSS | |
| Human Serum Albumin Binder (ALB11002) | |
| GFTFSSFGMS or SFGMS | 18 |
| Human Serum Albumin Binder (ALB11002) (CDR1) | |
| (amino acids 6-10 of SEQ ID NO: 18) | |
| SISGSGSDTLYADSVKG or SISGSGSDTL | 19 |
| (Human Serum Albumin Binder (ALB11002) CDR2) | |
| (amino acids 1-10 of SEQ ID NO: 19) | |
| GGSLSR | 20 |
| Human Serum Albumin Binder (ALB11002)(CDR3) | |
| MRFPSIFTAVLFAASSALAAPVNTTTEDETAQIPAEAVIGYSDLEGDF | 21 |
| DVAVLPFSNSTNNGLLFINTTIASIAAKEEGVSLEKR | |
| (Signal peptide) | |
| AMHVAQPAVVLASSRGIASFVCEYASPGKATEVRVTVLRQADSQVT | 22 |
| EVCAATYMMGNELTFLDDSICTG | |
| TSSGNQVNLTIQGLRAMDTGLYICKVELMYPPPYYLGIGNGTQIYVID | |
| PEPCPDSDFHHHHHHHHHGGQ | |
| (Human CTLA4 Amino Acid Sequence) | |

In an embodiment of the disclosure, the ISVD is ALB11002 which binds to human serum albumin.

The present disclosure includes a trivalent V_{HH} based CTLA4 binder comprising an HSA binder of the disclosure, for example, having the same combination of CDRs (i.e., CDR1, CDR2 and CDR3) as are present in ALB11002 or in a binder comprising the sequence of SEQ ID NO: 17. See Table 2.

Optionally, ALB11002 lacks the C-terminal Alanine. In an embodiment of the disclosure, the HSA binder comprises the amino acid sequence of SEQ ID NO: 17 but including a mutation at position 1, 1, 89, 110 or 112, e.g., comprising a set of mutations set forth in Table 2 herein.

Residue 1 of SEQ ID NO: 17 can be D or E. If residue 1 is D, the HSA binder may be designated as 1D and if residue 1 is E, the HSA binder may be designated as 1E.

The present disclosure includes HSA binders comprising one, two or three of the CDRs of a HSA binder wherein each comprises 0, 1, 2, 3, 4, 5, 6, 7, 8, 9 or 10 amino acid substitutions, e.g., conservative substitutions, and/or comprises 100, 99, 98, 97, 96 or 95% sequence identity relative to the CDRs that are in the HSA binder sequences set forth of Table 2 or are set forth in SEQ ID NOs: 67-69, wherein an HSA binder having such CDRs retains the ability to bind to HSA.

In an embodiment of the disclosure, the half-life extender is an anti-HSA ISVD (e.g., a sdAb) comprising:
a CDR1 that comprises the amino acid sequence GFTFSSFGMS (SEQ ID NO: 18);
and
a CDR2 that comprises the amino acid sequence SISGSGSDTL (SEQ ID NO: 19); and
a CDR3 that comprises the amino acid sequence GGSLSR (SEQ ID NO: 20);
   and, optionally, having:
a degree of sequence identity with the amino acid sequence of SEQ ID NO: 17 (in which any C-terminal extension that may be present as well as the CDRs are not taken into account for determining the degree of sequence identity) of at least 85%, preferably at least 90%, more preferably at least 95% (in which the CDRs, any C-terminal extension that may be present, as well as the mutations at positions 1, 11, 89, 110 and/or 112 required by the specific aspect involved are not taken into account for determining the degree of sequence identity):
   and/or
no more than 7, such as no more than 5, preferably no more than 3, such as only 3, 2 or 1 “amino acid differences” (as defined herein, and not taking into account any of the above-listed mutations at position(s) 1, 11, 89, 110 and/or 112 that may be present and not taking into account any C-terminal extension that may be present) with the amino acid sequence of SEQ ID NO: 17 (in which said amino acid differences, if present, may be present in the frameworks and/or the CDRs but are preferably present only in the frameworks and not in the CDRs);
   and optionally having:
a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

Again, as mentioned, such a serum albumin binding ISVD, when present, may contain within its sequence one or more framework mutations that reduce binding by pre-existing antibodies. In particular, when such a serum albumin binding ISVD is a V_{HH} antibody or a (single) domain antibody that is, essentially consist of and/or is derived from a V_{H} domain, the serum albumin binding ISVD may contain (a suitable combination of) amino acid residues/mutations at positions 11, 89, 110 and/or 112 that are as described in PCT/EP2015/060643 (WO2015/173325) or WO2017087588 and/or that essentially are as described herein for the CTLA4 binders of the disclosure. For example, PCT/EP2015/060643 (WO2015/173325) describes a number of variants of Alb-1, Alb-8 and Alb-23 that contain amino acid residues/mutations at positions 11, 89, 110 and/or 112 that reduce binding by pre-existing antibodies that can be used in the CTLA4 binders of the disclosure.

Generally, when a trivalent V_{HH} based CTLA4 binder of the disclosure has increased half-life (e.g. through the presence of a half-life increasing ISVD or any other suitable way of increasing half-life), the resulting trivalent V_{HH} based CTLA4 binder preferably has a half-life (as defined herein) that is at least 2 times, preferably at least 5 times, for example at least 10 times or more than 20 times, greater than the half-life of the monovalent CTLA4 antibody binder (as measured in either in man and/or a suitable animal model, such as mouse or cynomolgus monkey). In particular, a CTLA4 binder of the disclosure preferably has a half-life (as defined herein) in human subjects of at least 1 day, preferably at least 3 days, more preferably at least 7 days, such as at least 10 days.

As will be clear to the skilled person, when a trivalent V_{HH} based CTLA4 binder is intended for systemic administration and/or for prevention and/or treatment of a chronic disease or disorder, it will usually be preferred that said trivalent V_{HH} based CTLA4 binder has increased half-life (as defined herein). More preferably, such a trivalent V_{HH} based CTLA4 binder will contain a half-life extending ISVD such as, preferably, an ISVD and in particular a V_{HH} based CTLA4 binder binding to human serum albumin (as described herein).

### Formulations

In some aspects of the disclosure, the formulations described herein minimize the formation of antibody aggregates (high molecular weight species) and particulates, high and low molecular weight species, minimize oxidation of methionine residues, and ensure that the antibody, single domain antibody, nanobody, or multivalent V_{HH} retains biological activity over time.

In one aspect, the disclosure includes various formulations of a trivalent V_{HH} based CTLA4 binder. For example, the present disclosure includes formulations comprising (i) a trivalent V_{HH} based CTLA4 binder, (ii) a buffer (e.g., L-histidine or acetate), (iii) a non-reducing sugar (e.g., sucrose); (iv) a non-ionic surfactant (e.g., polysorbate 80); and (v) an antioxidant (e.g., L-methionine).

In one aspect, the formulation further comprises an anti-PD-1 antibody. In another aspect, the formulation may further comprise a chelator. In one embodiment, the chelator is diethylenetriaminepentaacetic acid (DTPA).

In one aspect, the disclosure also includes various co-formulations of a trivalent V_{HH} based CTLA4 binder and an anti-human PD-1 antibody, or antigen binding fragment thereof. In one embodiment, the present disclosure includes formulations comprising (i) a trivalent V_{HH} based CTLA4 binder, (ii) an anti-human PD-1 antibody or antigen binding fragment thereof, (iii) a buffer (e.g., L-histidine or acetate), (iv) a non-reducing sugar (e.g., sucrose), (v) a non-ionic surfactant (e.g., polysorbate 80), and (vi) an antioxidant (e.g., L-methionine). In one embodiment, the formulation may further comprise a chelator (e.g., DTPA).

Pharmaceutical formulations described herein may include buffers. The term “buffer” encompasses those agents which maintain the solution pH of the liquid formulations described herein in an acceptable range, or, for lyophilized formulations described herein, provide an acceptable solution pH prior to lyophilization and/or after reconstitution.

Buffers that are useful in the pharmaceutical formulations and methods of the disclosure include succinate (sodium or potassium), L-histidine, phosphate (sodium or potassium), Tris (tris (hydroxymethyl)aminomethane), diethanolamine, citrate (sodium), acetate (sodium) and the like. In an embodiment of the disclosure, buffer is present in the formulation at a concentration of about 1-20 mM (1, 2, 3, 4, 5, 6, 7, 8, 9, 10, 11, 12, 13, 14, 15, 16, 17, 18, 19 or 20 mM). In specific embodiments of the disclosure, the buffer is histidine buffer. In another embodiment, the buffer is L-histidine buffer. In specific embodiments of the disclosure, the buffer is sodium acetate. In specific embodiments of the disclosure, the buffer is sodium acetate trihydrate. In specific embodiments of the disclosure, the sodium acetate trihydrate is present at a concentration of about 0.86 mg/mL.

In another embodiment, the formulation comprises acetic acid at a concentration of about 10 mM. In specific embodiments of the disclosure, the acetic acid is present at a concentration of about 0.22 mg/mL.

In one embodiment, the buffer has a pH in the range from about 4.5 to about 6.0. In another embodiment, the pH is in the range from about 5.0-6.0. In another embodiment, the pH is in the range from about 4.5-5.6. In another embodiment, the pH is about 5.0. In arriving at the exemplary formulation, histidine and acetate buffers in the pH range of 4.4-7.4 were explored for suitability and showed a pH range of 4.5-5.6 showed lower aggregation levels at accelerated temperatures. When a range of pH values is recited, such as “a pH between pH 5.5 and 6.0,” the range is intended to be inclusive of the recited values. For example, a range from about 5.0 to about 6.0 includes 5.0, 5.1, 5.2, 5.3, 5.4, 5.5, 5.6, 5.7, 5.8, 5.9, and 6.0. For lyophilized formulations, unless otherwise indicated, the pH refers to the pH after reconstitution. pH is typically measured at 25° C. using standard glass bulb pH meter. As used herein, a solution comprising “acetate buffer at pH X” refers to a solution at pH X and comprising the acetate buffer, i.e., the pH is intended to refer to the pH of the solution.

In certain embodiments, the formulation may be a co-formulation of a trivalent V_{HH} based CTLA4 binder and an anti-human PD-1 antibody wherein the concentration of the anti-human PD-1 antibody is higher than that of the trivalent V_{HH} based CTLA4 binder (weight based), and the pH of the co-formulation is about 5.0.

In an embodiment of the disclosure, the formulation comprises a non-reducing sugar. As used herein, “non-reducing sugar” is a sugar not capable of acting as a reducing agent because it does not contain or cannot be converted to contain a free aldehyde group or a free ketone group. Examples of non-reducing sugars include but are not limited to disaccharides such as sucrose and trehalose. In an embodiment, the non-reducing sugar is present in an amount of from about 1-16% (w/v) (1, 2, 3, 4, 5, 6, 7, 8, 9, 11, 12, 13, 14, 15, or 16%). In another embodiment, the non-reducing sugar is present in an amount from about 6% to about 8% (w/v) (6, 7, or 8%). In a further embodiment, the non-reducing sugar is present in an amount of about 6% (w/v). In a further embodiment, the non-reducing sugar is present in an amount of about 7% (w/v). In a further embodiment, the non-reducing sugar is present in an amount of about 8% (w/v). In one embodiment, the non-reducing sugar sucrose, trehalose, or raffinose. In another embodiment, the non-reducing sugar is sucrose. In a further embodiment, the sucrose is present at 6-8% w/v. In one embodiment, the sucrose is present at 6% (w/v). In one embodiment, the sucrose is present at 7% (w/v). In one embodiment, the sucrose is present at 8% (w/v).

The formulations described herein also comprise a surfactant. As used herein, a surfactant is a surface active agent that is amphipathic in nature. Surfactants may be added to the formulations herein to provide stability, reduce and/or prevent aggregation or to prevent and/or inhibit protein damage during processing conditions such as purification, filtration, freeze-drying, transportation, storage, and delivery. In one aspect of the disclosure, a surfactant may be useful for providing additional stability to the active ingredient(s).

Non-ionic surfactants that may be useful in the formulations and co-formulations described herein include, but are not limited to, polyoxyethylene sorbitan fatty acid esters (Polysorbates, sold under the trade name Tween® (Uniquema Americas LLC, Wilmington, DE)) including Polysorbate-20 (polyoxyethylene sorbitan monolaurate), Polysorbate-40) (polyoxyethylene sorbitan monopalmitate), Polysorbate-60 (polyoxyethylene sorbitan monostearate), and Polysorbate-80) (polyoxyethylene sorbitan monooleate): polyoxyethylene alkyl ethers such as Brij® 58 (Uniquema Americas LLC, Wilmington, DE) and Brij® 35; poloxamers (e.g., poloxamer 188): Triton® X-100 (Union Carbide Corp., Houston, TX) and Triton® X-114; NP40; Span 20, Span 40, Span 60, Span 65, Span 80 and Span 85: copolymers of ethylene and propylene glycol (e.g., the Pluronic® series of nonionic surfactants such as Pluronic® F68, Pluronic® 10R5, Pluronic® F108, Pluronic® F127, Pluronic® F38, Pluronic® L44, Pluronic® L62 (BASF Corp., Ludwigshafen, Germany); and sodium dodecyl sulfate (SDS). In one embodiment, the non-ionic surfactant is polysorbate 80 or polysorbate 20. In one embodiment, the non-ionic surfactant is polysorbate 20. In another embodiment, the non-ionic surfactant is polysorbate 80.

The amount of non-ionic surfactant to be included in the formulations of the disclosure is an amount sufficient to perform the desired function, i.e., a minimal amount necessary to stabilize the active pharmaceutical ingredient (i.e., the trivalent V_{HH} based CTLA4 binder, or both the trivalent V_{HH} based CTLA4 binder and the anti-human PD-1 antibody or antigen binding fragment thereof) in the formulation. All percentages listed for polysorbate 80 are % w/v. Typically, the surfactant is present in a concentration of from about 0.008% to about 0.1% w/v. In some embodiments of this aspect of the disclosure, the surfactant is present in the formulation in an amount from about 0.01% to about 0.1%; from about 0.01% to about 0.09%; from about 0.01% to about 0.08%; from about 0.01% to about 0.07%; from about 0.01% to about 0.06%; from about 0.01% to about 0.05%; from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03% w/v. In specific embodiments, the surfactant is present in an amount of about 0.02% w/v. In alternative embodiments, the surfactant is present in an amount of about 0.01%, about 0.015%, about 0.025%, about 0.03%, about 0.035%, or about 0.04% w/v.

In exemplary embodiments of the disclosure, the surfactant is a nonionic surfactant selected from the group consisting of: Polysorbate 20 and Polysorbate 80. In preferred embodiments, the surfactant is Polysorbate 80.

In specific embodiments, the formulations of the disclosure comprise about 0.01% to about 0.04% w/v polysorbate 80. In further embodiments, the formulations described herein comprise polysorbate 80 in an amount of about 0.008% w/v, about 0.01% w/v. In one embodiment, the amount of polysorbate 80 is about 0.015 w/v %. In another embodiment, the amount of polysorbate 80 is about 0.02% w/v. In a further embodiment, the amount of polysorbate 80 is about 0.025% w/v. In another embodiment, the amount of polysorbate 80 is about 0.03% w/v. In a further embodiment, the amount of polysorbate 80 is about 0.035% w/v. In another embodiment, the amount of polysorbate 80 is about 0.04% w/v. In a further embodiment, the amount of polysorbate 80 is about 0.045% w/v. In particular embodiments, the formulations of the disclosure comprise about 0.02% w/v polysorbate 80.

Certain embodiments of the formulations of the present disclosure also comprise methionine, or a pharmaceutically acceptable salt thereof. In one embodiment, the methionine is L-methionine. In another embodiment, the methionine is a pharmaceutically acceptable salt of L-methionine, such as, for example, methionine HCl. In an embodiment, methionine is present in the formulation at a concentration of about 1-20 mM (1, 2, 3, 4, 5, 6, 7, 8, 9, 10, 11, 12, 13, 14, 15, 16, 17, 18, 19 and 20 mM). In another embodiment, the methionine is present from about 5 mM to about 10 mM (5, 6, 7, 8, 9 and 10 mM). In another embodiment, the methionine is present at about 10 mM.

The formulations described herein may further comprise a chelating agent. In an embodiment of the disclosure, chelating agent is present in the formulation at a concentration of about 1-50 μM (e.g., 1, 5, 10, 15, 20, 25, 30, 35, 40, 45, or 50 μM). In one embodiment, the chelating agent is DTPA. In another embodiment, the chelating agent is EDTA. In some additional embodiment, the DTPA is the antioxidant which can be present in any of the following amounts 1, 5, 10, 15, 20, 25, 30, 35, 40, 45, or 50 μM in any of the formulations described herein.

### Lyophilized Compositions

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e., in a small volume) if necessary, for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP), i.e., in anticipation of reconstitution in a low volume of water. Subsequent water or isotonic buffer can then readily be used to dilute the DP to a lower concentration. Typically, excipients are included in a lyophilized formulation of the present disclosure at levels that will result in a roughly isotonic formulation when reconstituted at high DP concentration, e.g., for subcutaneous administration. Reconstitution in a larger volume of water to give a lower DP concentration will necessarily reduce the tonicity of the reconstituted solution, but such reduction may be of little significance in non-subcutaneous, e.g., intravenous, administration. If isotonicity is desired at lower DP concentration, the lyophilized powder may be reconstituted in the standard low volume of water and then further diluted with isotonic diluent, such as 0.9% sodium chloride.

The lyophilized formulations of the present disclosure are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about-30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hours). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the present disclosure are reconstituted prior to administration. The protein may be reconstituted to a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. In one embodiment, the protein concentration after reconstitution is about 10-300 mg/mL. In one embodiment, the protein concentration after reconstitution is about 20-250 mg/mL. In one embodiment, the protein concentration after reconstitution is about 150-250 mg/mL. In one embodiment, the protein concentration after reconstitution is about 180-220 mg/mL. In one embodiment, the protein concentration after reconstitution is about 50-150 mg/mL. In one embodiment, the protein concentration after reconstitution is about 100 mg/mL. In one embodiment, the protein concentration after reconstitution is about 75 mg/mL. In one embodiment, the protein concentration after reconstitution is about 50 mg/mL. In one embodiment, the protein concentration after reconstitution is about 25 mg/mL. High protein concentrations are particularly useful where subcutaneous delivery of the reconstituted formulation is intended. However, for other routes of administration, such as intravenous administration, lower concentrations of the protein may be desired (e.g., from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g., phosphate-buffered saline), sterile saline solution, Ringer's solution or dextrose solution.

### Liquid Compositions

A liquid antibody formulation can be made by taking the drug substance which is in liquid form (e.g., trivalent V_{HH} based CTLA4 binder in an aqueous formulation) and buffer exchanging it into the desired buffer as the last step of the purification process. There is no lyophilization step in this embodiment. The drug substance in the final buffer is concentrated to a desired concentration. Excipients such as sucrose and polysorbate 80 are added to the drug substance and it is diluted using the appropriate buffer to final protein concentration. The final formulated drug substance is filtered using 0.22 μm filters and filled into a final container (e.g. glass vials).

### III. Methods of Use

The disclosure also relates to a method of treating cancer in a subject, the method comprising administering an effective amount of any of the formulations of the disclosure, i.e., any formulation described herein, to the subject. In some specific embodiments of this method, the formulation is administered to the subject via intravenous administration. In other embodiments, the formulation is administered to the subject by subcutaneous administration. In one embodiment a method is provided for treating cancer in a human patient comprising administering any formulation of the disclosure to the patient.

In any of the methods of the disclosure, the cancer can be selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g. hormone refractory prostate adenocarcinoma), pancreatic cancer, colon cancer, esophageal cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma.

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In one embodiment, a method is provided for treating metastatic non-small cell lung cancer (NSCLC) in a human patient comprising administering a formulation of the disclosure to the patient. In specific embodiments, the patient has a tumor with high Tumor Proportion Score (TPS)≥50%) and was not previously treated with platinum-containing chemotherapy. In other embodiments, the patient has a tumor with TPS≥1% and was previously treated with platinum-containing chemotherapy. In still other embodiments, the patient has a tumor with TPS≥1% and was not previously treated with platinum-containing chemotherapy. In specific embodiments, the patient had disease progression on or after receiving platinum-containing chemotherapy. In certain embodiments, the patient's tumor has no EGFR or ALK genomic aberrations. In certain embodiments, the patient's tumor has an EGFR or ALK genomic aberration and had disease progression on or after receiving treatment for the EGFR or ALK aberration(s) prior to receiving the formulation.

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments, the cancer is selected from the group consisting of melanoma, non-small cell lung cancer, relapsed or refractory classical Hodgkin lymphoma, head and neck squamous cell carcinoma, urothelial cancer, esophageal cancer, gastric cancer, and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

Additional cancers that can benefit from treatment with the formulations described herein include those associated with persistent infection with viruses such as human immunodeficiency viruses, hepatitis viruses class A, B and C. Epstein Barr virus, human papilloma viruses that are known to be causally related to for instance Kaposi's sarcoma, liver cancer, nasopharyngeal cancer, lymphoma, cervical, vulval, anal, penile and oral cancers.

The formulations can also be used to prevent or treat infections and infectious disease. Thus, a method is provided for treating chronic infection in a mammalian subject comprising administering an effective amount of a formulation of the disclosure to the subject. In some specific embodiments of this method, the formulation is administered to the subject via intravenous administration. In other embodiments, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C. Epstein Barr virus, human cytomegalovirus, human papilloma viruses, and herpes viruses. Viral infections with hepatitis B and C and HIV are among those considered to be chronic viral infections.

The formulations disclosed herein may be administered to a patient in combination with one or more “additional therapeutic agents”. The additional therapeutic agent may be a biotherapeutic agent (including but not limited to antibodies to VEGF, EGFR, Her2/neu. VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods provided herein, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist.

Suitable routes of administration may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g., subcutaneous administration) may require a lyophilized formulation to enable reconstitution at high concentration.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g., biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK: Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32: Lipsky et al. (2000) New Engl. J. Med. 343:1594-1602: Physicians' Desk Reference 2017 (Physicians' Desk Reference. 71st Ed); Medical Economics Company: ISBN: 9781563638381: 71st edition (2017). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See. e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See. e.g., Yang et al. (2003) New Engl. J. Med. 349:427-434; Herold et al. (2002) New Engl. J. Med. 346:1692-1698: Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

Embodiments of the disclosure also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of an CTLA4 related disease; (g) halting or delaying the progression cancer; (h) stabilization of CTLA4 related disease; (i) inhibiting the growth or survival of tumor cells; (j) eliminating or reducing the size of one or more cancerous lesions or tumors; (k) reduction of the progression, onset or severity of CTLA4 related disease; (l) reducing the severity or duration of the clinical symptoms of CTLA4 related disease such as cancer (m) prolonging the survival of a patient relative to the expected survival in a similar untreated patient n) inducing complete or partial remission of a cancerous condition or other CTLA4 related disease, o) treatment of cancer; or p) treatment of chronic infections.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology. Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA: de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290: Wright et al. (2000) Immunity 13:233-242: Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742: Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetry (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the present disclosure.

Having described different embodiments of the disclosure herein with reference to the accompanying drawings, it is to be understood that the disclosure is not limited to those precise embodiments, and that various changes and modifications may be effected therein by one skilled in the art without departing from the scope or spirit of the disclosure as defined in the appended claims.

### EXAMPLES

### Materials

Trivalent V_{HH} based CTLA4 binder (F023700912) referenced in table 1 & 2. If applicable, two different viable formulation buffers (sodium acetate with methionine and histidine) in the presence of sucrose, and polysorbate 80 were utilized. Lead buffer system: (25 mg/ml of F023700912 in 10 mM sodium acetate pH 5.0 buffer and contains 10 mM L-methionine, 0.02% (w/v) polysorbate 80 (PS-80), and 8% (w/v) sucrose). Back up buffer system: 25 mg/ml of F023700912 in 10 mM histidine pH 5.5 buffer and contains 0.02% (w/v) polysorbate 80 (PS-80), and 8% (w/v) sucrose). For some examples, only trivalent V_{HH} based CTLA4 binder with appropriate buffer for evaluation was utilized.

### Example 1—Trivalent V_{HH} Based CTLA4 Binder High-Throughout Formulation Screening

High-throughput pre-formulation developability studies were performed on transient material of F023700912 to evaluate: biophysical/biochemical liabilities and pre-formulation (pH, salt, and buffer) conditions. Thirty different conditions including multiple variables such as buffer species (Acetate, Histidine, and Phosphate), pH (ranging from 4.4-7.4), and ionic strength (0-150 mM NaCl). The concentration of F023700912 was kept constant at 1 mg/mL. The samples were analyzed by UV/Vis spectrophotometry for turbidity (A350) as measurement of colloidal stability; size exclusion chromatography (UP-SEC) to detect the formation of high molecular weight species; capillary isoelectric focusing (cIEF) to measure the effect of stress on distribution of charges at the surface of the molecule: reduced sodium dodecyl sulfate capillary electrophoresis (CE-SDS) to detect proteolytic cleavage; and flow cytometry analysis to detect sub-visible aggregates.

A high throughput buffer-pH-excipient screen (pre-formulation developability study) was performed for F023700912 and the stability of the molecule was monitored under accelerated temperature conditions. F023700912 was found to be stable in 10 mM Acetate or 10 mM Histidine buffer in the pH range of 5.0-6.0.

### Example 1a—pH Ranging and Buffer Selection

Based on the high throughput pre-formulation development, a pH ranging study with acetate, histidine and citrate buffer and the addition of F023700912 was performed. Buffers were selected to cover pH range of 4.5-5.8. F023700912 was dialyzed in each of these buffers and filtered after adjusting the concentration to achieve target concentration of 25 mg/mL. Stress samples were placed at 50° C. for 10 days and the controls were placed at 5° C. The samples were analyzed for monomer content loss by UP-SEC and change in charge variants by HP-IEX and fragmentation by Caliper assay.

pH ranging studies were conducted to determine the optimal pH for the final formulation. 25 mM buffer was added as a variable to evaluate impact of buffer strength on the stability of the molecule. The pH was varied within +0.5 units from the target pH of 5.0.

Table 3 shows the analytical summary for the key assays for F023700912 formulation following thermal stress at different pH conditions and in the presence of acetate, citrate, and histidine buffers. The pH of the final formulation was measured following the dialysis in the appropriate buffers and no pH adjustments were made after dialysis.

The stressed samples for histidine and citrate buffers all turned turbid and were not submitted for analysis. Only three out of four acetate buffer formulations were analyzed to compare the pH dependent behavior. The data showed that F023700912 favored acetate as compared to histidine or citrate. F023700912 exhibited improved stability at a pH range around 5.0 and hence pH range of 4.9 to 5.2 was evaluated further to establish pH set point.

Additionally, to minimize the rate of deamidation, pH 5.0 was selected.

TABLE 3
pH Ranging Study for 25 mg/mL, F023700912

| | | 10 mM Acetate | | | | 10 mM Citrate | | | 10 mM His | | |
|---|---|---|---|---|---|---|---|---|---|---|---|
| | | pH | pH | pH | pH | pH | pH | pH | pH | pH | pH |
| Assay | | 4.5 | 4.9 | 5.2 | 5.5 | 5.2 | 5.5 | 5.8 | 5.2 | 5.5 | 5.8 |
| Turbitidy | Control | 0.036 | 0.041 | 0.052 | 0.043 | 0.049 | 0.044 | 0.045 | 0.045 | 0.046 | 0.046 |
| (A350-A500) | Stress | 0.165 | 0.375 | 2.108 | | 0.231 | 0.241 | 0.240 | 2.493 | | |
| | Δ | | | | | | | | | | |
| | Turbidity | | | | | | | | | | |
| UPSEC (%) | | | | | | | | | | | |
| HMW | Control | 0.49 | 0.51 | 0.56 | 0.59 | 0.55 | 0.59 | 0.65 | 0.5 | 0.5 | 0.52 |
| MAIN | | 99.5 | 99.5 | 99.4 | 99.3 | 99.4 | 99.4 | 99.3 | 99.4 | 99.4 | 99.4 |
| LMW | | ND | ND | ND | 0.1 | 0.05 | 0.05 | 0.05 | 0.08 | 0.07 | 0.06 |
| HMW | Stress | 36.47 | 34.99 | 24.66 | | | | | | | |
| MAIN | | 61.8 | 63.8 | 74.3 | | | | | | | |
| LMW | | 1.73 | 1.24 | 1.05 | | | | | | | |
| | Δ % | 36.0 | 34.5 | 24.1 | | | | | | | |
| | HMW | | | | | | | | | | |
| IEX (%) | | | | | | | | | | | |
| ACIDIC | Control | 67.26 | 67.34 | 67.27 | 67.29 | 67.12 | 67.21 | 67.53 | 69.2 | 69.48 | 69.78 |
| MAIN | | 30.4 | 30.4 | 30.4 | 30.4 | 30.5 | 30.5 | 30.5 | 28.3 | 28.4 | 28.3 |
| LMW | | 2.29 | 2.22 | 2.33 | 2.32 | 2.33 | 2.32 | 1.95 | 2.46 | 2.15 | 1.94 |
| ACIDIC | Stress | 67.87 | 69.71 | 71.63 | | | | | | | |
| MAIN | | 22.3 | 21.4 | 19.8 | | | | | | | |
| LMW | | 9.82 | 8.88 | 8.58 | | | | | | | |
| | Δ % | 8.1 | 9.0 | 10.6 | | | | | | | |
| | Main | | | | | | | | | | |
| | Peak | | | | | | | | | | |
| CESDS (%) | | | | | | | | | | | |
| MAIN | Control | 97.6 | 97.72 | 97.62 | 97.59 | 97.62 | 97.53 | 97.57 | 97.54 | 97.47 | 97.68 |
| MAIN | Stress | 92.49 | 93.19 | 94.46 | | | | | | | |
| | Δ % | 5.1 | 4.5 | 3.2 | | | | | | | |
| | Main | | | | | | | | | | |
| | Peak | | | | | | | | | | |

### Example 1b—Buffer Strength and Impact of Sucrose

For buffer strength evaluation, 25 mg/mL F023700912 in 25 mM, 50 mM, 75 mM acetate buffer each were prepared at target pH of 4.8. 8% sucrose was added to two of the formulations as shown in Table 4. For excipient selection, pH 4.8 was chosen as a target set point and buffers with 25 mM and 50 mM strength were chosen as base buffers with sucrose as stabilizer. The samples were stressed at 50° C. for 3 days. The samples were analyzed for turbidity, sub-visible particulates by microflow imaging (MFI), monomer content loss by UP-SEC and change in charge variants by HP-IEX.

In order to evaluate impact of buffer strength and sucrose, formulations with different buffer strengths (25 mM, 50 mM and 75 mM) at target pH of 4.8 were stressed at elevated temperatures. To evaluate the impact of sucrose as a stabilizer, two formulations containing 8% sucrose were also coupled with this study. Table 4 is a summary of the data comparing the impact of thermal stress on these formulations. F023700912 stability improved as a function of sucrose concentration. A sucrose ranging study was performed and there was a direct correlation between the amount of sucrose and % HMW.

TABLE 4
Buffer Strength Evaluation for 25 mg/mL, F023700912

| | | pH 4.8 | | | | pH 5.0 |
|---|---|---|---|---|---|---|
| | | | | | 50 mM + | 75 mM + |
| Assay | | 25 mM | 50 mM | 75 mM | 8% Suc | 8% Suc |
| Turbidity | Control | 0.035 | 0.041 | 0.041 | 0.047 | 0.042 |
| (A350-A500) | Stress | 0.182 | 0.484 | 1.039 | 0.156 | 0.517 |
| | Δ Turbidity | 0.144 | 0.443 | 0.998 | 0.109 | 0.475 |
| UPSEC (%) | | | | | | |
| HMW | Control | 0.9 | 0.9 | 0.9 | 0.8 | 0.9 |
| MAIN | | 99.2 | 99.1 | 99.1 | 99.2 | 99.1 |
| LMW | | <QL | <QL | <QL | <QL | <QL |
| HMW | Stress | 20.9 | 40.4 | 56.6 | 14.8 | 16.0 |
| MAIN | | 78.9 | 59.3 | 43.1 | 84.8 | 83.0 |
| LMW | | 0.3 | 0.3 | 0.3 | 0.4 | 1.0 |
| | Δ % HMW | 20.0 | 39.5 | 55.8 | 14.0 | 15.1 |
| IEX (%) | | | | | | |
| ACIDIC | Control | 67.75 | 67.6 | 67.54 | 67.88 | 67.9 |
| MAIN | | 30.3 | 30.4 | 30.5 | 30.1 | 30.2 |
| LMW | | 1.91 | 1.99 | 2 | 2.05 | 1.85 |
| ACIDIC | Stress | 65.04 | 65.22 | 65.74 | 65.71 | 66.14 |
| MAIN | | 28 | 28.4 | 28 | 27.2 | 26.7 |
| LMW | | 6.94 | 6.41 | 6.29 | 7.13 | 7.21 |
| | Δ % Main | 2.3 | 2.0 | 2.5 | 2.9 | 3.5 |
| | Peak | | | | | |
| CESDS (%) | | | | | | |
| MAIN | Control | 98.1 | 98.0 | 98.2 | 98.0 | 97.9 |
| LMW | | 1.8 | 1.8 | 1.7 | 1.9 | 2.0 |
| MAIN | Stress | 96.6 | 96.4 | 96.6 | 97.2 | 96.3 |
| LMW | | 3.3 | 3.5 | 3.1 | 2.8 | 3.6 |
| | Δ % Main | 1.5 | 1.7 | 1.6 | 0.8 | 1.6 |
| | Peak | | | | | |

TABLE 5
F023700912 Stability and Sucrose Concentration,

| | |
|---|---|
| T 0 | 50° C. 10 days |

| | HMW | Main | HMW | Main |
|---|---|---|---|---|
| 0% Suc | 0.4 | 99.3 | 18.8 | 79.5 |
| 4% Suc | 0.3 | 99.3 | 11.2 | 87.0 |
| 8% Suc | 0.3 | 99.3 | 7.6 | 90.4 |
| 12% Suc | 0.4 | 99.3 | 6.1 | 91.9 |
| 16% Suc | 0.4 | 99.2 | 4.8 | 93.1 |
| 20% Sucrose | 0.5 | 99.2 | 4.3 | 93.5 |

Table 5 shows the rate of decrease in formation of HMW, which provides increased stability, as a function of increased sucrose concentration. The sucrose addition leads to F023700912 compaction and also reduced acetate F023700912 formulation interactions. Taken together with Example 5, the NMR data suggests that F023700912 formulation is stabilized indirectly by sucrose exclusion leading to preferential hydration that strongly affects linker motions.

### Example 1c—Formulation Selection

In order to determine optimum pH, a separate pH optimization study was carried out at various pH's including 4.7, 5.0, 5.3 and 5.6 in acetate buffer at specific concentrations specified in the table below, and pH 4.7 in histidine buffer with target concentration of 25 mg/mL. All formulations contained 8% w/v sucrose. The samples were stressed at 50° C. for 8 days. The samples were analyzed for turbidity, sub-visible particulates by microflow imaging (MFI), monomer content loss by UP-SEC and change in charge variants by HP-IEX.

It was observed that higher buffer strength led to decreased stability for the trivalent V_{HH} based CTLA4 binder. Out of the three strengths tested, 25 mM was found to be most stable. Sucrose had a positive impact on stability of the molecule, specifically in preventing aggregate formation and is a feature in some of the formulations. The data were further analyzed to establish pH set point and to optimize the buffer strength for the formulation.

In analysis of the data, 10 mM Histidine buffer performed better than 25 mM acetate buffer. Also, pH 5.0 seemed to be a stable center point where the turbidity as well as aggregate formation was minimized.

TABLE 6
Formulation Comparison, F023700912

| | | 10 mM | | | | 25 mM | | 10 mM |
|---|---|---|---|---|---|---|---|---|
| | | Acetate | | | | Acetate | | His |
| | | pH | pH | pH | pH | pH | pH | pH |
| Assay | | 4.7 | 5.0 | 5.3 | 5.6 | 4.7 | 5.3 | 4.7 |
| Turbidity | Control | 0.035 | 0.036 | 0.04 | 0.038 | 0.039 | 0.043 | 0.045 |
| (A350-A500) | Stress | 0.058 | 0.064 | 0.4 | 0.238 | 0.11 | 0.238 | 0.196 |
| | Turbidity | 0.023 | 0.028 | 0.06 | 0.2 | 0.071 | 0.195 | 0.151 |
| UPSEC (%) | | | | | | | | |
| HMW | Control | 0.9 | 0.9 | 0.9 | 1.0 | 0.9 | 0.9 | 0.9 |
| MAIN | | 99.1 | 99.1 | 99.1 | 99.0 | 99.1 | 99.1 | 99.1 |
| LMV | | <QL | <QL | <QL | <QL | <QL | <QL | <QL |
| HMW | Stress | 6.9 | 6.2 | 7.2 | 6.1 | 13.0 | 8.7 | 6.8 |
| MAIN | | 92.2 | 92.9 | 92.1 | 93.1 | 85.9 | 90.5 | 92.4 |
| LMV | | 0.94 | 0.83 | 0.75 | 0.79 | 1.12 | 0.81 | 0.78 |
| | Δ % HMW | 6.0 | 5.3 | 6.3 | 5.1 | 12.1 | 7.8 | 5.9 |
| IEX (%) | | | | | | | | |
| ACIDIC | Control | 67.3 | 67.3 | 67.3 | 67.2 | 67.0 | 67.0 | 68.7 |
| MAIN | | 29.6 | 29.5 | 29.6 | 29.7 | 29.8 | 29.9 | 28.3 |
| BASIC | | 3.2 | 3.2 | 3.1 | 3.1 | 3.2 | 3.1 | 3.0 |
| ACIDIC | Stress | 67.5 | 69.1 | 70.7 | 74.5 | 67.1 | 70.2 | 74.6 |
| MAIN | | 22.8 | 21.7 | 20.1 | 17.2 | 22.6 | 20.3 | 17.0 |
| BASIC | | 9.7 | 9.3 | 9.2 | 8.3 | 10.3 | 9.5 | 8.4 |
| | Δ % Main | 6.8 | 7.8 | 9.5 | 12.5 | 7.2 | 9.6 | 11.3 |
| | Peak | | | | | | | |
| CESDS (%) | | | | | | | | |
| MAIN | Control | 97.6 | 97.8 | 97.7 | 97.6 | 97.7 | 97.5 | 97.8 |
| LMW | | 2.2 | 2.1 | 2.2 | 2.2 | 2.1 | 2.4 | 2.1 |
| MAIN | Stress | 95.4 | 96.4 | 96.6 | 96.6 | 95.9 | 96.5 | 96.6 |
| LMW | | 4.0 | 3.4 | 3.3 | 3.3 | 4.0 | 3.4 | 3.3 |
| | Δ % Main | 2.2 | 1.4 | 1.1 | 1.0 | 1.8 | 1.0 | 1.2 |
| | Peak | | | | | | | |

### Example 1d—Evaluation of L-Methionine as an Excipient

Buffer system: 25 mg/mL of F023700912 in 10 mM sodium acetate pH 5.0 buffer, 0.02% (w/v) polysorbate 80 (PS-80), and 8% (w/v) sucrose).

During a light stress study (using only visible light of ICH equivalent at 4000 Lux), it was observed that the formulation exposed to visible light turned yellow due to oxidation. L-methionine was evaluated as a potential excipient to decrease the rate of oxidation and prevent color change. Nitrogen overlay was also pursued as a potential solution and a combined study was performed. Formulations, with and without methionine and nitrogen overlay, were exposed to 0.2× and 0.5×ICH equivalent of visible light stress and then samples were visibly compared to color change with appropriate dark controls. All the data are summarized in Table 7.

It was observed that addition of methionine as well as nitrogen overlay individually prevented color change in the formulation at 0.2×. However, at 0.5×, only the formulation containing methionine and nitrogen overlay was completely clear. Formulations containing methionine and nitrogen overlay as well as their combination performed much better than base formulation. The 0.2×ICH equivalent is close to real manufacturing and handling condition.

TABLE 7
Evaluation of L-Methionine as Excipient

| | 0.05X | | 0.1X | | 0.2X | | | | 0.5X |
|---|---|---|---|---|---|---|---|---|---|
| | −M | +M | −M | +M | −M | +M | +N2 | +M + N2 | −M |
| Visual | — | — | — | — | Clear, | Clear, | Clear, | Clear, | Clear, |
| | | | | | Slight | Color | Color | Color | Deep |
| | | | | | Yellow, | Less, | Less, | Less, | Yellow, |
| | | | | | FOP | FOP | FOP | FOP | FOP |
| Turbidity | 0.048 | 0.040 | 0.057 | 0.046 | 0.136 | 0.055 | 0.047 | 0.045 | 1.055 |
| UPSEC | | | | | | | | | |
| % HMW | 1.0 | 1.0 | 1.1 | 1.0 | 1.3 | 1.1 | 1.1 | 1.0 | 8.1 |
| % Main | 99.0 | 99.0 | 98.9 | 99.0 | 98.7 | 98.9 | 98.9 | 98.9 | 91.5 |
| % LMW | 0.0 | 0.0 | 0.0 | 0.0 | 0.0 | 0.0 | 0.0 | 0.1 | 0.4 |
| IEX | | | | | | | | | |
| % Acidic | 67.7 | 67.6 | 67.2 | 67.6 | 67.1 | 67.4 | 67.5 | 67.6 | 62.6 |
| % Main | 28.8 | 28.7 | 28.6 | 28.6 | 27.6 | 28.5 | 28.9 | 28.7 | 19.9 |
| % Basic | 3.5 | 3.6 | 4.2 | 3.8 | 5.3 | 4.1 | 3.6 | 3.7 | 17.5 |
| R-CESDS | | | | | | | | | |
| % Main | 97.7 | 98.1 | 97.8 | 98.6 | 97.6 | 97.9 | 97.9 | 98.5 | 42.9 |
| % LMW | 2.2 | 1.9 | 2.0 | 1.3 | 2.1 | 1.6 | 1.9 | 1.4 | 3.6 |

| | 0.5X | | | DC-0.5X | | | |
|---|---|---|---|---|---|---|---|
| | +M | +N2 | +M + N2 | −M | +M | +N2 | +M + N2 |
| Visual | Clear, | Clear, | Clear, | — | — | — | — |
| | Slight | Slight | Color | | | | |
| | Yellow, | Yellow, | Less, | | | | |
| | FOP | FOP | FOP | | | | |
| Turbidity | 0.181 | 0.089 | 0.056 | 0.036 | 0.037 | 0.036 | 0.035 |
| UPSEC | | | | | | | |
| % HMW | 1.5 | 1.3 | 1.2 | 0.9 | 0.9 | 0.9 | 0.9 |
| % Main | 98.5 | 98.6 | 98.7 | 99.0 | 99.1 | 99.0 | 99.1 |
| % LMW | 0.1 | 0.1 | 0.1 | 0.0 | 0.0 | 0.0 | 0.0 |
| IEX | | | | | | | |
| % Acidic | 65.8 | 67.3 | 67.0 | 67.5 | 67.6 | 67.4 | 67.6 |
| % Main | 25.9 | 27.9 | 28.2 | 29.2 | 29.2 | 29.2 | 29.1 |
| % Basic | 8.3 | 4.8 | 4.8 | 3.3 | 3.2 | 3.4 | 3.3 |
| R-CESDS | | | | | | | |
| % Main | 97.3 | 94.8 | 97.8 | 97.6 | 98.4 | 98.4 | 98.6 |
| % LMW | 2.7 | 1.6 | 2.1 | 1.1 | 1.5 | 1.3 | 1.2 |

Note:
+M = addition of 10 mM Methionine, +N2 = +5% O2 = nitrogen overlay with final oxygen level around 5%. +M + N2 = with 10 mM Methionine and nitrogen overlay.

### Example 2

### Surfactant Ranging Study

Buffer System: 25 mg/mL of F023700912 in 10 mM sodium acetate pH 5.0 buffer and contains mM L-methionine, and 8% (w/v) sucrose).

Surfactant ranging studies were carried out to determine level of surfactant needed for stabilization of formulation upon agitation stress. PS-80 is a commonly used surfactant in therapeutic protein formulations. In the surfactant ranging study, susceptibility of F023700912 to agitation stress was assessed in the presence and absence of PS-80. Range of 0.05 to 0.5 mg/mL for PS-80 was evaluated. The data are summarized in Table 8.

In the absence of surfactant, samples remained clear suggesting inherent stability of the molecule against agitation stress. There was no change in any biochemical attributes as measured by UPSEC, IEX and CESDS. Only changes in subvisible particles were observed.

There were higher numbers of subvisible particles in formulations devoid of surfactant. There was a dramatic decrease in subvisible particles in formulations containing PS80.

TABLE 8
Surfactant Ranging Study

| | RT Control | | | 300 RPM | | | | |
|---|---|---|---|---|---|---|---|---|
| | 0 | 0.2 | 0.5 | 0 | 0.05 | 0.1 | 0.2 | 0.5 |
| TURBIDITY | 0.034 | 0.034 | 0.037 | 0.034 | 0.033 | 0.033 | 0.034 | 0.035 |
| SUBVISIBLE | | | | | | | | |
| PARTICLES | | | | | | | | |
| ≥2 | 2303 | 299 | 1161 | 8464 | 83 | 1186 | 2754 | 530 |
| ≥5 | 427 | 42 | 232 | 2437 | 21 | 246 | 567 | 120 |
| ≥10 | 67 | 7 | 14 | 549 | 14 | 42 | 42 | 53 |
| ≥25 | 3 | 0 | 3 | 78 | 3 | 3 | 0 | 7 |
| ≥50 | 0 | 0 | 0 | 21 | 0 | 0 | 0 | 0 |
| UPSEC (%) | | | | | | | | |
| HMW | 0.8 | 0.8 | 0.8 | 0.8 | 0.8 | 0.8 | 0.8 | 0.8 |
| MAIN | 99.1 | 99.1 | 99.1 | 99.1 | 99.1 | 99.1 | 99.1 | 99.1 |
| LMW | 0.1 | 0.1 | 0.1 | 0.1 | 0.1 | 0.1 | 0.1 | 0.1 |
| IEX (%) | | | | | | | | |
| ACIDIC | 69.6 | 69.7 | 69.7 | 69.6 | 69.7 | 69.8 | 69.7 | 69.7 |
| MAIN | 28.8 | 28.8 | 28.8 | 28.7 | 28.7 | 28.7 | 28.8 | 28.7 |
| BASIC | 1.6 | 1.6 | 1.6 | 1.6 | 1.6 | 1.6 | 1.6 | 1.6 |
| R-CESDS (%) | | | | | | | | |
| LMW | 0.5 | 0.6 | 0.6 | 0.7 | 0.7 | 0.6 | 0.7 | 0.7 |
| MAIN | 99.5 | 99.4 | 99.4 | 99.3 | 99.3 | 99.4 | 99.3 | 99.3 |
| PS80 | 0.04 | 0.19 | 0.45 | 0.02 | 0.04 | 0.08 | 0.19 | 0.41 |

### Example 3

### Freeze Thaw Stability

Freeze-thaw stability of F023700912 acetate formulation was evaluated. Formulation with 10 mM Acetate buffer, pH 5.0, 8% Sucrose, 10 mM L-Methionine and 0.02% w/v PS80 was evaluated for freeze thaw stability. Formulation showed no change upon 8× freeze-thaw as compared to the control that was stored at 5° C. (Table 9). The sub visible particles, turbidity and biochemical profiles were comparable after freeze-thaw. Overall, freeze-thaw cycling had no measurable impact on aggregation or biochemical properties of lead formulation.

TABLE 9
Freeze Thaw Study

Freeze-Thaw

| | Control | 3X | 5X | 8X |
|---|---|---|---|---|
| Turbidity | 0.033 | 0.034 | 0.034 | 0.032 |
| Subvisible Particles | | | | |
| ≥2 | 500 | 413 | 810 | 338 |
| ≥5 | 81 | 44 | 53 | 33 |
| ≥10 | 14 | 10 | 5 | 7 |
| ≥25 | 0 | 0 | 0 | 0 |
| ≥50 | 0 | 0 | 0 | 0 |
| UPSEC (%) | | | | |
| HMW | 0.4 | 0.4 | 0.4 | 0.4 |
| MAIN | 99.6 | 99.5 | 99.6 | 99.6 |
| LMW | 0.1 | 0.1 | 0.1 | 0.1 |
| IEX (%) | | | | |
| ACIDIC5 | 15.7 | 15.6 | 16.0 | 15.9 |
| ACIDIC4 | 31.5 | 31.5 | 31.7 | 31.6 |
| ACIDIC3 | 7.4 | 7.4 | 7.4 | 7.4 |
| ACIDIC2 | 3.2 | 3.2 | 3.1 | 3.2 |
| ACIDIC1 | 14.1 | 14.1 | 13.8 | 13.9 |
| MAIN | 27.1 | 27.1 | 27.0 | 27.0 |
| BASIC1 | 1.1 | 1.1 | 1.0 | 1.0 |
| % TOTAL ACIDIC | 71.9 | 71.8 | 72.0 | 71.9 |
| % MAIN | 27.1 | 27.1 | 27.0 | 27.0 |
| % TOTAL BASIC | 1.1 | 1.1 | 1.0 | 1.1 |
| R-CESDS (%) | | | | |
| LMW | 0.8 | 0.8 | 0.7 | 0.6 |
| MAIN | 99.2 | 99.3 | 99.3 | 99.4 |

### Example 4

This study compares the stability of F023700912 in two different viable formulation buffers (sodium acetate with methionine and histidine) in the presence of sucrose, and polysorbate 80.

This study compares the stability of two formulations:
Formulation A: 25 mg/mL protein concentration of F023700912 in 10 mM sodium acetate pH 5.0, 8% sucrose, 10 mM Methionine and 0.20 mg/mL PS-80,
Formulation B: 25 mg/mL protein concentration of F023700912 in 10 mM histidine pH 5.5, 8% sucrose, and 0.20 mg/mL PS-80.

The two formulations were staged for up to 6 months at 5° C., 25° C. and 40° C. and were analyzed for turbidity, sub-visible particulates by MFI, UP-SEC and HP-IEX. The data are summarized in Table 10 and Table 11

### 6 Month Stability in Acetate Buffer

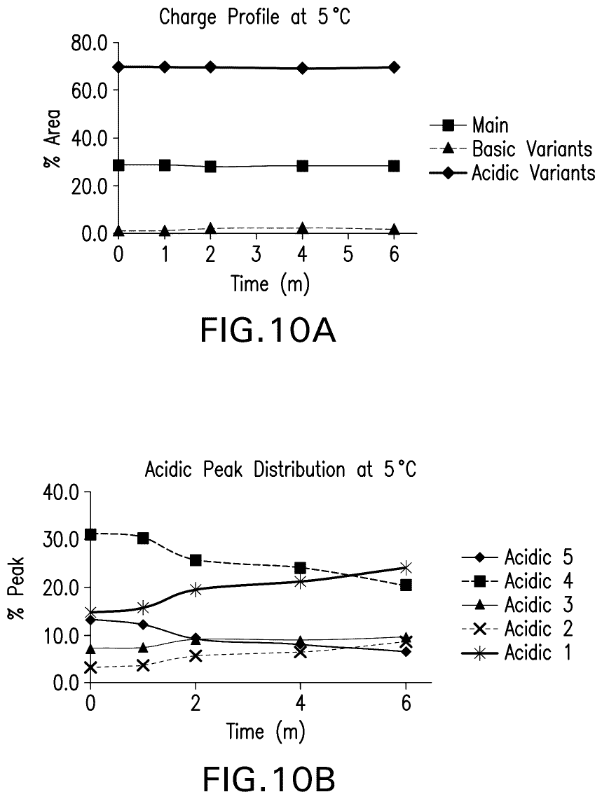

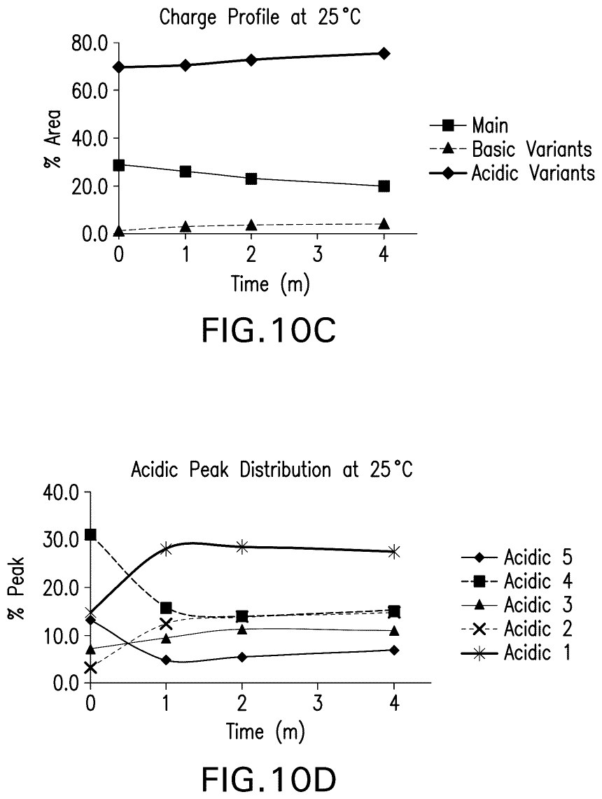

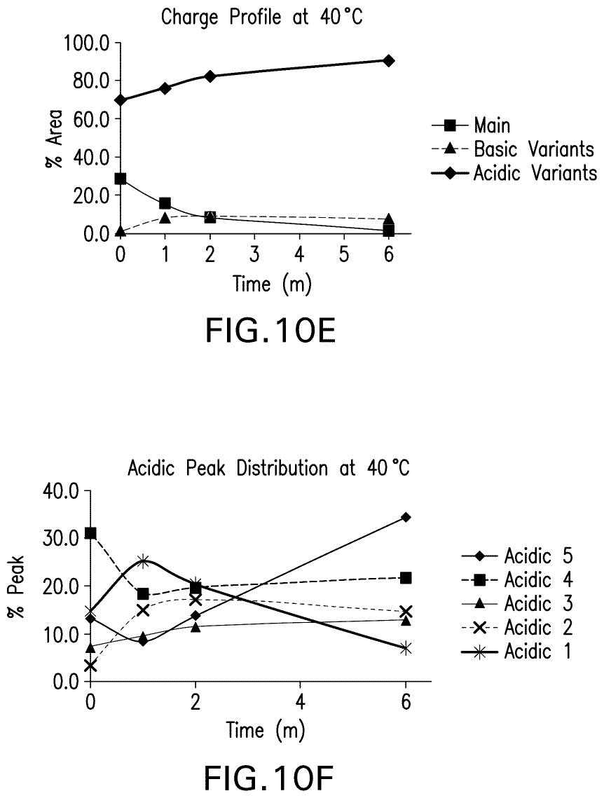

No significant changes were observed in the UP-SEC and HP-IEX at the recommended storage temperature (2-8° C.) for acidic variants, main and basic variants for up to 6 months. A slight increase in the individual acidic peaks (acidic 1 and 2) was observed with a corresponding decrease in acidic peaks 4 and 5 at the recommend storage condition [FIGS. 10; A and B]. This shift in individual peaks can be explained by the equilibrium between deamidated N74/N228 and the succinimide intermediate.

At the accelerated condition (25° C.), a slight increase in acidic variants and a corresponding decrease in main peak were observed. Increases in acidic peaks (acidic variants) 1 and 2 are observed due to two factors: deamidation of main peak as well as conversion of deamidated form (acidic peaks 4 and 5) to succinimide intermediate (acidic peaks 1 and 2) [FIGS. 10; C and D], supporting the deamidation mechanism. No significant changes were observed by UP-SEC during course of stability study.

At the stressed condition (40° C.), a significant increase in acidic variants and or corresponding decrease of the main peak was observed after storage at 6 months [FIG. 10; E and F]. This increase in acidic variants and decrease in main at the accelerated and stressed temperatures is expected as deamidation is temperature dependent.

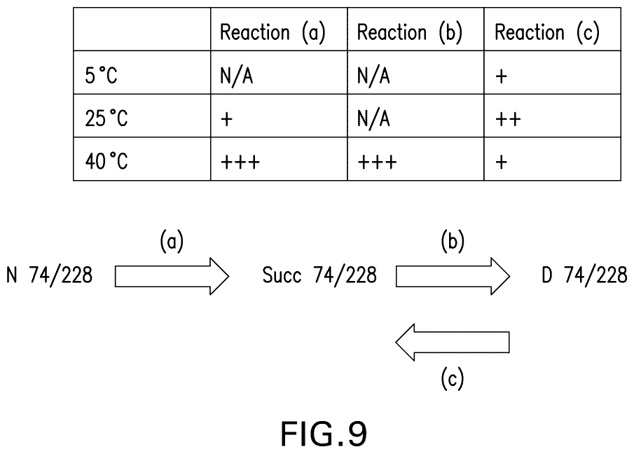

The results from these stability studies highlight three reactions occurring simultaneously at different rates as a function of temperature: (a) Deamidation of F023700912 by formation of the corresponding succinimide species (b) conversion of succinimide intermediate into fully deamidated species and (c) conversion of fully deamidated species to succinimide intermediate. At the recommended storage temperature (2-8° C.), reaction (c) is the predominant reaction while there is little to none of reaction (a) and (b). At 25° C., rate of reactions (a) and (c) increase with increase in temperature with little to no change in reaction (b). However, at 40° C., reaction (a) and (c) are predominant at earlier time-point but is exceeded by reactions (a) and (b) with progression of time. A simplified proposed mechanism for trivalent V_{HH} based CTLA4 binder deamidation is illustrated in FIG. 9.

TABLE 10
Early Research Stability Study (Acetate)

| | Initial | 5° C. | | | | 25° C. | | | 40° C. | | |
|---|---|---|---|---|---|---|---|---|---|---|---|
| | TO | 1 M | 2 M | 4 M | 6 M | 1 M | 2 M | 4 M | 1 M | 2 M | 6 M |
| Turbidity | 0.033 | 0.031 | 0.032 | 0.032 | 0.034 | 0.034 | 0.038 | 0.045 | 0.045 | 0.066 | 0.209 |
| Subvisible | | | | | | | | | | | |
| Particles | | | | | | | | | | | |
| (per mL) | | | | | | | | | | | |
| ≥2 | 714 | 617 | 1642 | 930 | 1427 | 1102 | 1253 | 789 | 2428 | 1388 | 9938 |
| ≥5 | 117 | 129 | 432 | 163 | 216 | 246 | 269 | 166 | 884 | 409 | 1048 |
| ≥10 | 10 | 17 | 44 | 12 | 44 | 35 | 19 | 5 | 163 | 88 | 172 |
| ≥25 | 0 | 5 | 0 | 0 | 0 | 3 | 3 | 0 | 3 | 0 | 7 |
| ≥50 | 0 | 0 | 0 | 0 | 0 | 0 | 0 | 0 | 0 | 0 | 0 |
| UPSEC (%) | | | | | | | | | | | |
| Total | 0.8 | 0.8 | 0.7 | 0.8 | 0.8 | 0.8 | 0.9 | 1.1 | 3.8 | 4.0 | 5.8 |
| HMWS | | | | | | | | | | | |
| Monomer | 99.2 | 99.3 | 99.3 | 99.1 | 99.0 | 98.6 | 98.1 | 97.3 | 94.2 | 90.6 | 65.2 |
| LMW | 0.1 | 0 | 0 | 0.1 | 0.3 | 0.6 | 1.0 | 1.6 | 2.0 | 5.4 | 29.1 |
| IEX % | | | | | | | | | | | |
| Acidic 5 | 13.2 | 12.3 | 9.4 | 8.2 | 6.6 | 4.8 | 5.4 | 7.1 | 8.4 | 13.7 | 34.3 |
| Acidic 4 | 31.2 | 30.4 | 25.8 | 24.2 | 20.6 | 15.9 | 14.0 | 15.3 | 18.4 | 19.6 | 21.8 |
| Acidic 3 | 7.3 | 7.5 | 9.2 | 9.0 | 9.6 | 9.5 | 11.3 | 10.9 | 9.4 | 11.4 | 12.8 |
| Acidic 2 | 3.3 | 3.8 | 5.7 | 6.5 | 8.6 | 12.4 | 13.8 | 14.7 | 14.8 | 17.0 | 14.6 |
| Acidic 1 | 14.8 | 15.7 | 19.5 | 21.3 | 24.1 | 28.1 | 28.5 | 27.5 | 25.1 | 20.3 | 6.9 |
| Main | 28.8 | 28.8 | 28.1 | 28.4 | 28.4 | 26.2 | 23.4 | 20.1 | 15.7 | 8.7 | 1.8 |
| BASIC 1 | 1.4 | 1.5 | 2.3 | 2.4 | 2.0 | 3.1 | 3.7 | 4.3 | 8.1 | 9.2 | 7.8 |
| Acidic | 69.8 | 69.7 | 69.7 | 69.2 | 69.6 | 70.6 | 72.9 | 75.6 | 76.1 | 82.1 | 90.4 |
| Variants | | | | | | | | | | | |
| Total | 28.8 | 28.8 | 28.1 | 28.4 | 28.4 | 26.2 | 23.4 | 20.1 | 15.7 | 8.7 | 1.8 |
| MAIN | | | | | | | | | | | |
| Basic | 1.4 | 1.5 | 2.3 | 2.4 | 2.0 | 3.1 | 3.7 | 4.3 | 8.2 | 9.2 | 7.8 |
| Variants | | | | | | | | | | | |
| R-CESDS % | | | | | | | | | | | |
| LMW | 0.5 | 0.0 | 0.0 | 0.7 | 1.0 | 0.6 | 0.2 | 2.6 | 1.9 | 2.3 | 19.2 |
| MAIN | 99.5 | 100.0 | 100.0 | 99.3 | 99.0 | 99.4 | 99.8 | 97.4 | 98.1 | 97.7 | 79.4 |

### 6 Month Stability in Histidine Buffer

At 5° C., there were no significant changes observed in HP-IEX profile over six month stability. However, interconversion was observed within the acidic variants (acidic peaks) where acidic (variants) 4 and 5 decreased over time whereas acidic variants 1 and 2 increased. No changes were observed in the UPSEC or CE-SDS.

At 25° C., the sample showed increased turbidity as well as loss of monomer content over 4 months. In HP-IEX, there was an increase in acidic variants along with decrease in the main peak over stability without any significant change in basic variants. However, within acidic variants, there was significant interconversion. Acidic variants 4 and 5 decreased at 1 month and then increased at subsequent timepoints where acidic variant 1 increased initially and then decreased whereas acidic variant 2 increased throughout four months.

At 40° C., histidine formulation was turbid and thus was not analyzed for subvisible particles at 2M and it showed high levels of aggregates. At 6M, the formulation turned significantly turbid and no further analysis was performed.

TABLE 11
Early Research Stability Study (Histidine)

| | Initial | 5° C. | | | | 25° C. | | | 40° C. | | |
|---|---|---|---|---|---|---|---|---|---|---|---|
| | TO | 1 M | 2 M | 4 M | 6 M | 1 M | 2 M | 4 M | 1 M | 2 M | 6 M |
| Turbidity | 0.038 | 0.036 | 0.036 | 0.04 | 0.04 | 0.045 | 0.057 | 0.097 | 0.132 | 0.271 | 2.046 |
| Subvisible | | | | | | | | | | | |
| Particles | | | | | | | | | | | |
| (per mL) | | | | | | | | | | | |
| ≥2 | 732 | 987 | 1734 | 565 | 2108 | 2497 | 1388 | 2578 | 2222 | | |
| ≥5 | 305 | 299 | 553 | 177 | 250 | 1039 | 565 | 925 | 782 | | |
| ≥10 | 122 | 65 | 99 | 49 | 39 | 315 | 163 | 230 | 399 | | |
| ≥25 | 12 | 10 | 0 | 5 | 7 | 44 | 0 | 14 | 12 | | |
| ≥50 | 0 | 3 | 0 | 0 | 0 | 0 | 0 | 5 | 0 | | |
| UPSEC % | | | | | | | | | | | |
| HMW | 0.8 | 0.8 | 0.7 | 0.8 | 0.8 | 0.9 | 0.9 | 1.3 | 4.4 | 4.7 | NT |
| MAIN | 99.2 | 99.2 | 99.3 | 99.1 | 98.9 | 98.6 | 98.2 | 97.3 | 93.7 | 90.7 | NT |
| LMW | 0.1 | 0.0 | 0.0 | 0.1 | 0.3 | 0.6 | 0.9 | 1.4 | 1.9 | 4.7 | NT |
| IEX % | | | | | | | | | | | |
| Acidic 5 | 14.5 | 13.9 | 11.7 | 11.7 | 10.0 | 8.5 | 10.3 | 13.6 | 13.4 | 21.5 | NT |
| Acidic 4 | 31.9 | 31.5 | 28.7 | 28.2 | 26.5 | 23.7 | 25.2 | 26.8 | 25.9 | 28.1 | NT |
| Acidic 3 | 7.1 | 7.3 | 8.9 | 8.3 | 9.1 | 8.4 | 9.2 | 8.9 | 8.1 | 9.2 | NT |
| Acidic 2 | 3.1 | 3.4 | 4.6 | 4.9 | 6.3 | 9.6 | 10.6 | 12.3 | 13.0 | 13.7 | NT |
| Acidic 1 | 14.0 | 14.6 | 17.1 | 17.8 | 19.9 | 23.7 | 22.8 | 21.0 | 20.4 | 14.1 | NT |
| Main | 28.1 | 28.0 | 27.0 | 26.8 | 26.6 | 23.4 | 18.8 | 14.1 | 11.7 | 5.6 | NT |
| BASIC 1 | 1.4 | 1.4 | 2.1 | 2.4 | 1.7 | 2.7 | 3.0 | 3.2 | 7.6 | 7.7 | NT |
| % Acidic | 70.5 | 70.5 | 70.9 | 70.9 | 71.7 | 74.0 | 78.2 | 82.6 | 80.7 | 86.8 | NT |
| Variants | | | | | | | | | | | |
| % Total | 28.1 | 28.0 | 27.0 | 26.8 | 26.6 | 23.4 | 18.8 | 14.1 | 11.7 | 5.6 | NT |
| MAIN | | | | | | | | | | | |
| % Basic | 1.4 | 1.4 | 2.1 | 2.4 | 1.7 | 2.6 | 3.0 | 3.2 | 7.6 | 7.7 | NT |
| Variants | | | | | | | | | | | |
| R-CESDS % | | | | | | | | | | | |
| LMW | 0.6 | 0.3 | 0.0 | 0.8 | 1.0 | 1.4 | 0.0 | 2.6 | 1.6 | 2.3 | NT |
| MAIN | 99.4 | 99.8 | 100.0 | 99.2 | 99.0 | 98.7 | 100.0 | 97.4 | 98.4 | 97.7 | NT |

Overall, both formulations were comparable at 5° C. but histidine was found to be inferior to acetate at 25° C. and 40° C.

Based upon the results obtained from high-throughput formulation screening, F023700912 was found to be more stable in acetate or histidine buffer within the pH range of pH 4.0 to 6.0. With additional pH ranging studies, buffer type and strength selection studies, 25 mg/mL F023700912 is preferably formulated in 10 mM sodium acetate buffer at a target pH of 5.0. 80 mg/mL sucrose was found be effective in reducing protein self-association and is a stabilizer in the formulation. 10 mM L-methionine was incorporated as an antioxidant in the formulation. Based on surfactant ranging studies 0.20 mg/mL PS-80 was selected as the final surfactant concentration in the formulation. F023700912 formulation is a liquid drug product as solution for injection with recommended storage at 2-8° C. The solution for injection drug product is at pH 5.0. An excess fill of liquid can ensure the recovery of the desired amount for administration.

F023700912 formulation was found to be stable under refrigerated conditions as well as under freeze-thaw conditions.

### Example 5

### Materials Specific to Example 5:

F023700912
Glacial acetic acid, sodium acetate trihydrate and sucrose procured from Sigma-Aldrich (St. Louis, MO).
Polysorbate 80 (PS80) purchased from Croda (Edison, NJ).

F023700912 was dialyzed overnight into acetate buffer at pH between 4.6 and 5.5 with polysorbate 80 and 0, 4, 8, 12, 16, or 20% sucrose.

Samples were filtered using 0.22 μm filter (EMD Millipore Burlington, MA) and used for NMR analysis. NMR studies were performed on a Bruker Avance HD 800 spectrometer equipped with a 5 mm TCI cryoprobe. 2D fingerprinting data as acquired using a 5 mm sample tube, 500 uL; all other data was acquired using a 3 mm sample tube, 160 uL volume.

F023700912 was investigated using NMR structural fingerprints, proton relaxation and translational self-diffusion. Structural fingerprints and relaxation measurements assess the behavior of individual atoms and protein residues and can be affected by inter and intra-molecular changes. Diffusion measurements are used to assess the bulk behavior of each molecule.

### Methods

### Profiling and Structural Fingerprinting

A simple ¹H NMR spectrum of formulated proteins provides a rapid, low resolution assessment of protein structural changes. Initial data consisted of a protein profile that uses a diffusion filter to reduce the NMR signal from sucrose and buffer molecules. In the diffusion filtered proton NMR profile, all proton-containing solution components can be identified by their ¹H NMR signals. In addition, protons from the protein backbone, side chain and linker can be identified by their distinctive chemical shifts. For concentrated, well-behaved proteins, ¹H, ¹³C sfHMQC data can be acquired for the protein methyl groups to give a higher resolution assessment of ligand binding and/or structural changes. The ¹H, ¹³C sfHMQC data sets in this study were collected using signal from the naturally abundant ¹³C isotope, without enrichment.

### Protein-Enhanced DOSY

Pulsed magnetic field gradient NMR experiments can be used to measure the translational diffusion of multiple molecules in complicated mixtures. Diffusion ordered spectroscopy NMR experiments (DOSY) are commonly used to separate the signals of each proton-containing solution component by its proton ¹H NMR signals and translational diffusion constant (Dt). The decay of the ¹H NMR signal for each molecule in a gradient magnetic field yields the translational diffusion constant for each molecule. For NMR measurements of formulated proteins, DOSY experiments can yield, simultaneously, the diffusion constants of the protein and other solution components such as PEG, sucrose, buffer and water; and the effects of solution viscosity can be deconvoluted. Diffusion NMR experiments are typically performed with a linear magnetic field gradient that varies from an initial value of 5% to a final value of 95%. A modified protein-enhanced DOSY experiment can be made by starting the initial gradient magnetic field at 30% which efficiently reduces the excipient and water signals leaving mostly the protein DOSY NMR signal.

### Carr-Purcell-Meiboom-Gill (CPMG) Proton R2

Proton relaxation measurements can be used to probe molecular motions ranging from nanosecond to millisecond timescales. A conventional CPMG proton-R2 measurement (effective VCPMG=500 s⁻¹) was modified with a diffusion filter to produce protein-enhanced R2 measurement that reduces the ¹H NMR signals from all non-protein buffer components [R2Dt ref]. At low protein concentration, proton-R2 relaxation rates are proportional to the molecule's volume (R2 αtc=4πηr³/kT) but are also strongly affected by motional anisotropy and intermolecular interactions. These anisotropic components can be qualitatively separated using a R1R2 parameter and, more recently, R2Dt correlations have been used to assess and differentiate intermolecular interactions and internal/external motions.

### Accelerated Stability Study

A 20 mg/ml sample of F023700912 with different levels of sucrose was exposed to accelerated temperature of 50° C. for 10 days and corresponding unstressed controls were stored at 5° C. for the duration of the study. The samples were analyzed using size exclusion chromatography and the high molecular weight (HMW) and main peaks were calculated by using Empower software (Waters Corporation, MA). The reported ΔHMW values are difference between HMW at accelerated temperature and 5° C. controls.

### Viscosity Measurements

Viscosity measurements were performed on F023700912 using m-VROC viscometer (Rheosense, San Ramon, CA). The instrument was equilibrated at 20° C. and all viscosity measurements were carried out at the same temperature. 50% glycerol was used as the standard (viscosity 5 cP). Each sample was measured 10 times and first four replicates were ignored. The last six measurements were averaged to provide viscosity value for one sample. Both placeboes as well as protein solutions were measured in similar manner.

### Results

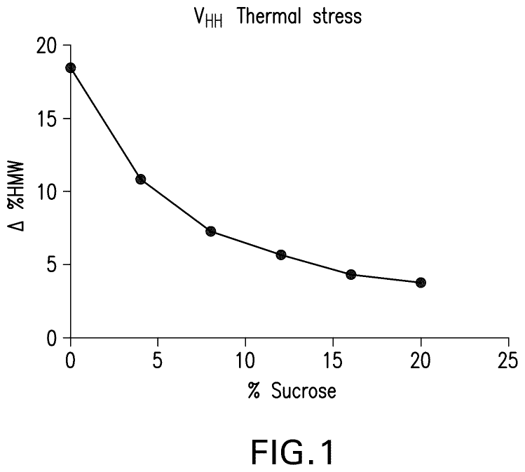

An accelerated stability study was carried out to evaluate the impact of increasing the level of sucrose on molecular stability. Increase in sucrose content resulted in decrease in HMW formation upon accelerated stress (FIG. 1). In absence of sucrose, the ΔHMW was highest at 18%. Sucrose addition lead to concentration dependent decrease in ΔHMW of 3.8% at 20% Sucrose concentration. FIG. 1 shows a high molecular weight species generation upon thermal stress of MV-V_{HH} at 50° C. for 10 days. Sucrose concentration ranges from 0 to 20%. The difference between stressed and unstressed samples is plotted as the ΔHMW

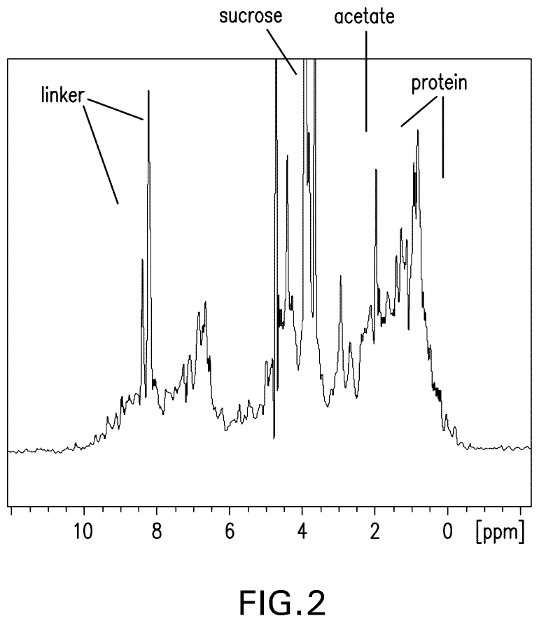

To understand the sucrose dependent changes in protein stability, the F023700912 structure and domain interactions were assessed using profiling and structural fingerprinting. In the ¹H NMR data, peaks originating from the folded domains of the trivalent V_{HH} based CTLA4 binder comprising three distinct modules, flexible linker (L1), buffer, and sucrose, FIG. 2, can be compared as a function of sucrose using 1D Profiling to provide a spectral difference measure which serves as a low-resolution structural assessment of any sucrose dependent changes to the protein. FIG. 2 shows the ¹H NMR spectrum of the trivalent V_{HH} based CTLA4 binder comprising three distinct modules and flexible linker(s). Peaks from different regions of the folded protein, flexible linker, excipients, water, and buffer can be detected and differentiated.

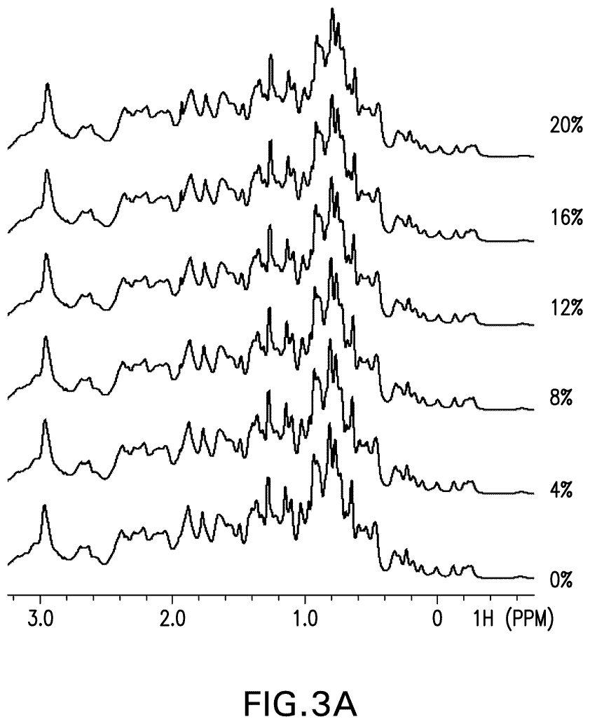

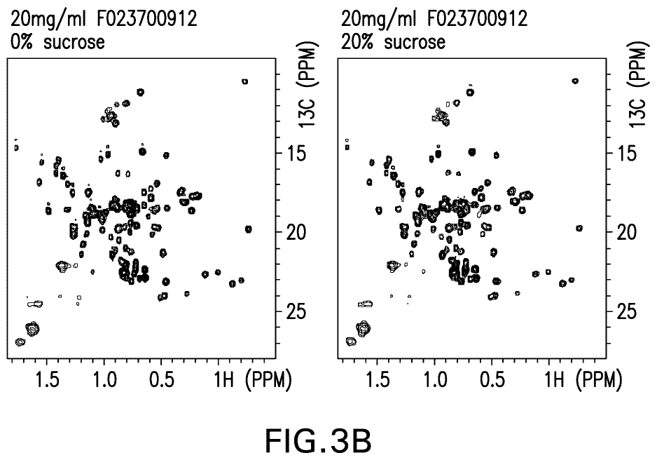

There were no direct protein-sucrose interactions detected using 2D NMR fingerprints. Protein spectra were compared with and without varying amounts of sucrose, and any site-specific binding in these experiments would be detected as peak shifts. While peak intensities are affected slightly by sucrose addition (consistent with viscosity differences), there is no evidence of direct sucrose-protein binding or protein conformational change. This was confirmed in higher resolution data from 1H-13C correlation spectroscopy (FIG. 3B) where no peak shifts were detected. No changes in FIG. 3A or FIG. 3B suggest the sucrose-dependent stabilization of the trivalent V_{HH} (antibody) is not a result of site-specific binding and does not result in detectable changes in structure or conformation.

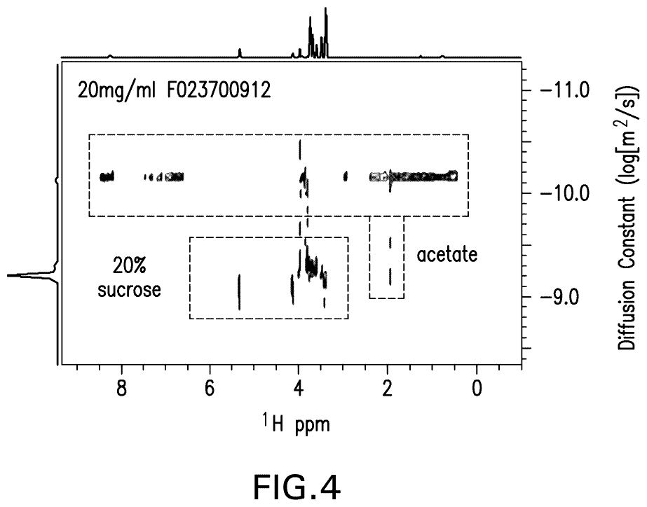

Interaction profiling was accomplished using a protein enhanced DOSY experiment, FIG. 4. The diffusion behavior of all protonated solution components can be monitored in situ under formulation conditions using a protein-enhanced DOSY experiment. First, translational self-diffusion data on F023700912 with 0% sucrose was collected. The detection dimension of this data set was dominated by protein peaks, but an acetate signal was also identified at 1.9 ppm. From the F023700912 diffusion data a hydrodynamic radius, Rh, of 3.9 nm was calculated; compared to aCTLA4 fragments which had a hydrodynamic radius of 1.65 nm. These data indicated that the F023700912 was in an extended conformation (expected Rh˜2.9 nm based on the molecular weight of the protein) which is consistent with the 2D ¹H, ¹³C sfHMQC fingerprinting data that suggests the V_{HH} domains are flexibly linked with minimal intramolecular interactions.

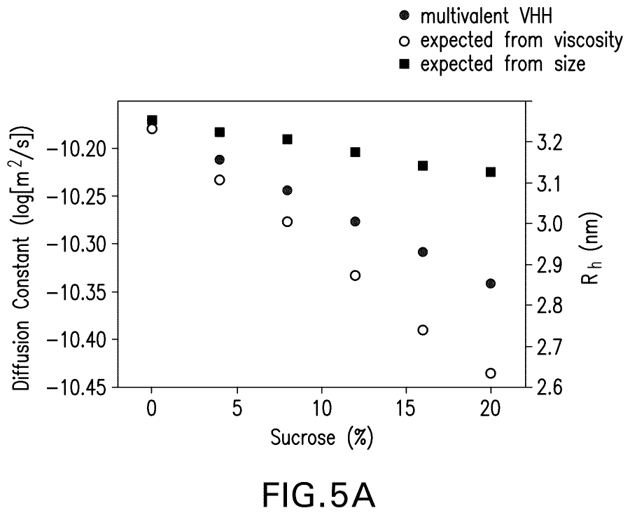

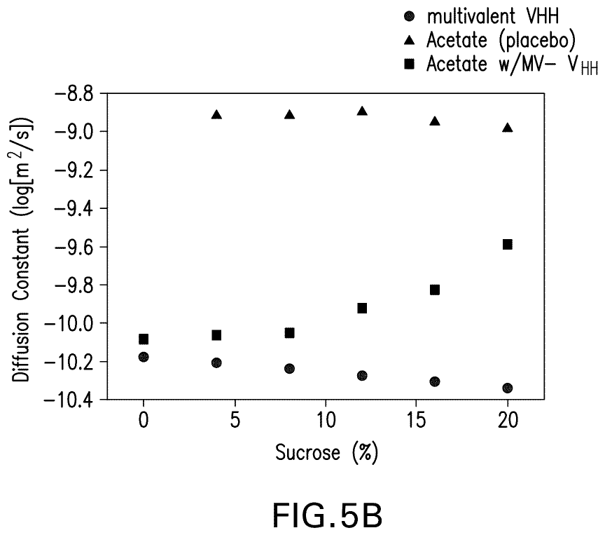

The effect of sucrose addition on the protein and buffer interactions was studied next using the protein-enhanced DOSY experiment on protein samples that contain 4%, 8%, 12% 16% and 20% sucrose. The effect of increasing sucrose concentration on sucrose self-diffusion can be assessed by monitoring the sucrose peak at 5.35 ppm while the effect of sucrose addition on F023700912 self-diffusion can be assessed by focusing on peaks in protein methyl region (1-0) ppm). As expected from changes in solution viscosity, the translational diffusion constants for both sucrose and the F023700912 decrease with increasing sucrose concentration. To determine the expected F023700912 diffusion behavior due to viscosity, a sucrose correction factor was calculated from the observed sucrose Dt measurements; based on the observation that the slope of diffusion versus sucrose concentration for sucrose in the absence and presence of the protein was the same, and then applied to the protein Dt measurements. This showed that F023700912 diffuses faster than expected based on the change in viscosity, FIG. 5A. Rh of F023700912 is directly calculated from the diffusion constants using the measured solution viscosity of F023700912 in sucrose. This showed a 20% decrease in Rh, from 3.3 nm at 0% sucrose to 2.6 nm at 20% sucrose, indicating compaction of the protein (FIG. 5A).

The diffusion behavior of acetate is dramatically different, FIG. 5B. In a solution of buffer only with no F023700912, acetate diffuses rapidly. With sucrose addition, its diffusion is slightly decreased due to the increased solution viscosity. In experiments on F023700912 in acetate buffer with no sucrose, acetate diffusion slows and nearly matches the protein diffusion rate which would be expected in cases where acetate interacts with the protein. The addition of sucrose increases the acetate diffusion rate; trending toward the free diffusion rate with increasing sucrose consistent with an increase in free acetate and reduced acetate-protein interactions.

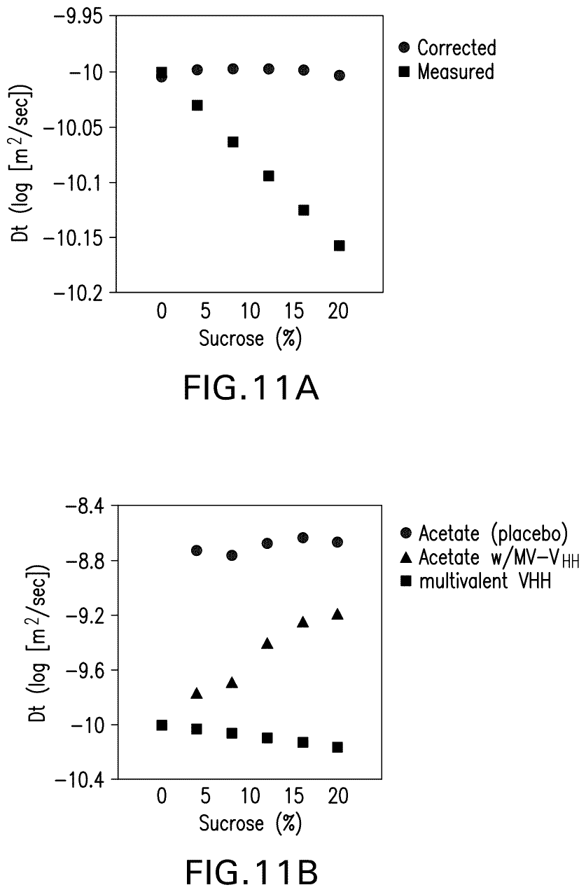

The sucrose viscosity correction factors used in the original diffusion data analysis were replaced with values determined from a calibration curve derived from sucrose NMR measurements. Protein diffusion data corrected for sucrose viscosity from a calibration curve showed that F023700914 diffuses at a constant rate. FIG. 11A, corresponding to an Rh of 3.7 nm for each protein-sucrose sample. No compaction was observed.

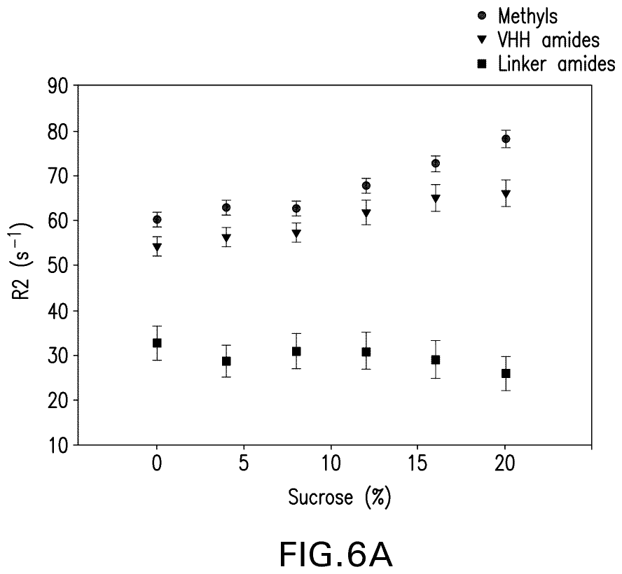

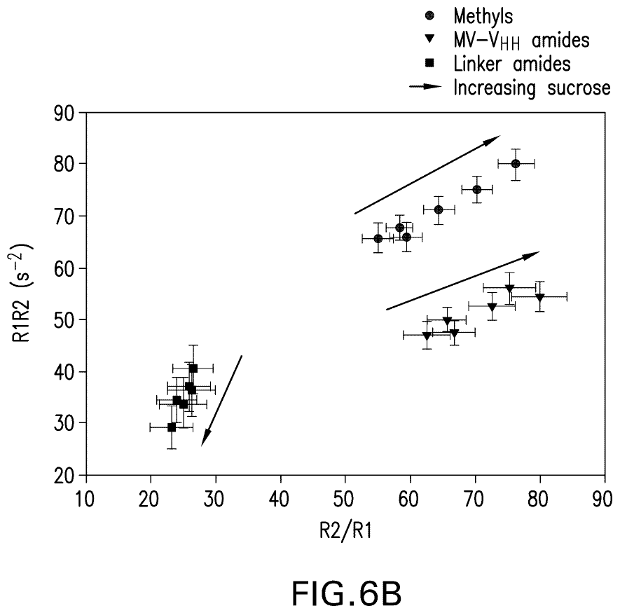

The effect of sucrose on protein motions was studied using protein-enhanced R1 and R2 relaxation measurements. ¹H NMR signals originate from atoms in different parts of the protein; motional changes to backbone, side chains and linker atoms can potentially be probed and differentiated. To compare the motions in different parts of the protein. R2 was measured for sidechain methyl and backbone amides, largely found in the core of the folded protein domains, as well as linker amides. FIG. 6A. An unanticipated inflection point is reached at ˜10% sucrose where F023700912 R2 starts increasing more dramatically. This could be due to either changes in protein motions or to viscosity since proton R2 rates are directly proportional to viscosity. The inflection also occurs in R1, R2 and in the viscosity independent parameter R1*R2, suggesting that this is a protein-related observation.

At 0% sucrose, the reduced linker R2 is consistent with greater flexibility than the methyl and proteins amide R2 which originate from the well-folded linked multivalent V_{HH} monomer domains. The linker R2 behavior is remarkable, trending lower with increasing amounts of sucrose. In these experiments, sucrose addition clearly influences protein motions, preferentially affecting linker atoms in R1 and R2 measurements.

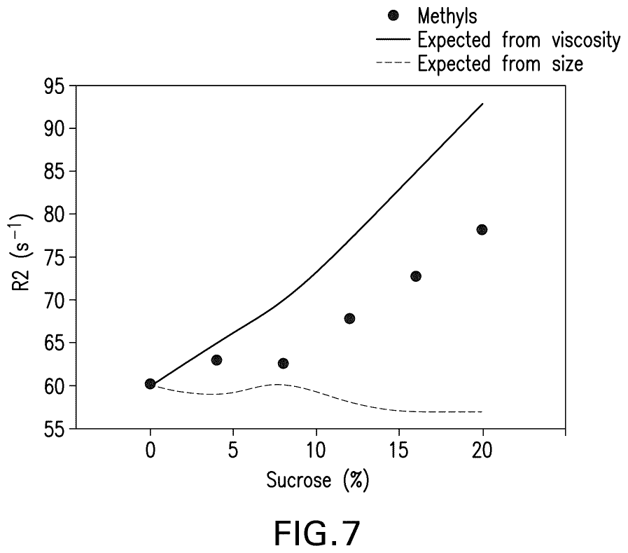

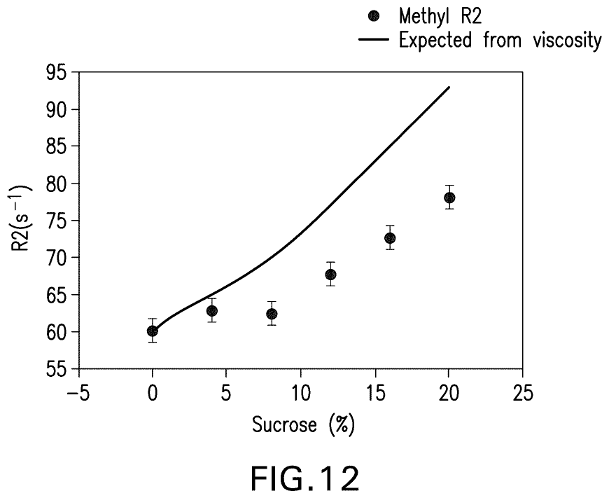

Comparing F023700912 R2 rates in the 0% and 20% sucrose data: the measured R2 data in the 20% sample are significantly lower (78 s⁻¹) than the anticipated 93 s⁻¹ expected from the 1.80-fold viscosity difference (assuming constant Rh), and significantly greater than the estimated 57 s⁻¹ expected based on the compaction of the protein (FIG. 7). This shows that viscosity and/or protein compaction do not fully explain the observed rotational motions of the F023700912, but that there is some other process modulating F023700912 motions on a (ms-us timescale) that is related to sucrose. The sucrose viscosity correction factors used in the original diffusion data analysis were replaced with values determined from a calibration curve derived from sucrose NMR measurements. FIG. 12 shows changes in motions in response to sucrose.

Additional experiments were then conducted to investigate the origin of the effects observed for sucrose addition. Factors involved in the modulation of acetate-protein interactions were examined. NMR can probe mechanisms including acetate displacement by sucrose-protein binding, competition from water due to increased hydration, protein aggregation, preferential sucrose-acetate interactions as well as indirect mechanisms.

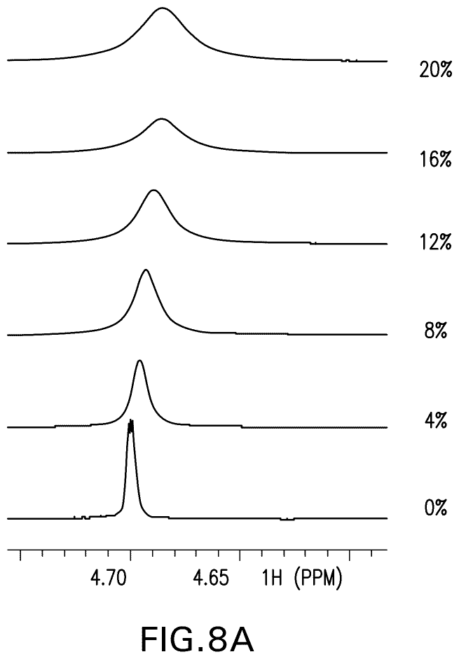

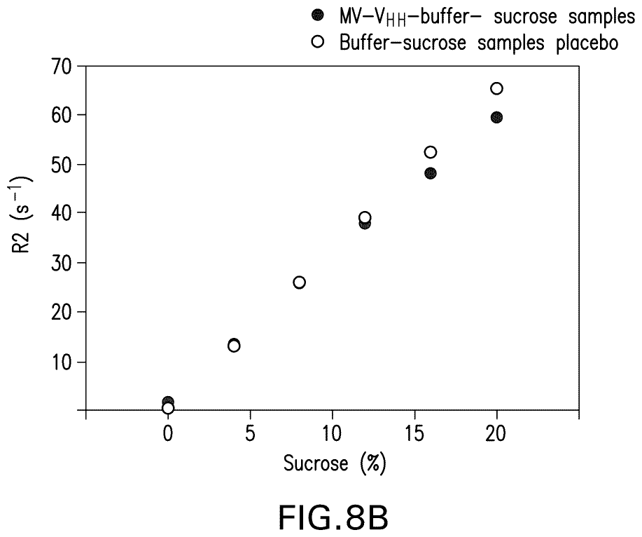

The prevailing mechanism for sucrose dependent protein stabilization is preferential exclusion, leading to protein hydration and stabilization. The expected change in protein hydration is challenging to directly measure experimentally due to rapid water diffusion; no change in protein-bound water would be anticipated. Measurements of directly bound waters molecules cannot typically be distinguished from bulk water, except in examples of water molecules that occupy internal cavities. Nonetheless we can use observed changes to water-sucrose interactions as a surrogate measurement for changes in water-protein interactions. The results of water R2 measurements are shown in FIG. 8A. While water protons are broadened by sucrose interactions, they exhibit very little broadening (R2 is less than 1 s⁻¹) in buffer alone and in buffer with protein. Differences in water R2 decay rates in sucrose-buffer solutions are compared to water R2 decay rates in sucrose-buffer-protein solutions. FIG. 8B. At 20% sucrose, the water R2 value is reduced by 5 s⁻¹ in the presence of protein. At 8% sucrose, the change in water R2 is less than 1 s⁻¹. The trend in FIG. 8B of lower than expected water R2 in the presence of sucrose suggests an overall reduction of sucrose-water interactions consistent with increased water-protein interactions associated with preferential protein hydration.

Multivalent molecules engineered from novel scaffolds can potentially bind to and crosslink multiple cell surface receptors to enhance signaling in the same cell; alternatively, different specificities can be selected to crosslink cells types. Multiple epitopes on the same target protein can potentially be engaged. And, finally, multivalent V_{HH} proteins potentially offer an engineering approach to combination therapies, delivering multiple therapeutic proteins with different mechanisms.

Combining novel protein scaffolds into multivalent therapeutic proteins will likely present different stability, developability and manufacturing challenges when compared with traditional mAbs. The sucrose-dependent stabilization of the trivalent V_{HH} based CTLA4 binder was analyzed through NMR spectroscopy. All molecules in the formulated protein sample can be studied together; protein-protein and protein-excipient interactions can be studied simultaneously. Solution NMR measurements are made in situ; formulated protein samples need not be diluted, frozen, heated or digested; no manipulation is necessary or desired. Measurements are made on the entire formulation (the mixture of the protein and excipients) rather than the active pharmaceutical ingredient (API: protein). NMR measurements uniquely measure changes to protein structure, conformation and dynamics.

NMR spectroscopy is used to assess protein-excipient interactions in therapeutic formulations. Structural profiling is used to capture both the protein's solution conformation and higher order structure details and as well as to probe for site specific interactions. Diffusion profiling is used to assess the protein Brownian motion as well as to delineate multi-component intermolecular interactions of all solution components. R2 measurements provided information on local motions and interactions.

A combination of NMR characterization techniques revealed CTLA4 binder modules of F023700912 to be flexibly linked with no observable domain interactions. No evidence of site-specific sucrose-protein interaction, no protein conformational changes and no changes to V_{HH}-V_{HH} interactions were detected. Sucrose addition produced multivalent Vum protein translational diffusion changes that were consistent with a decrease in hydrodynamic radius of the protein; direct evidence of compaction. Sucrose and acetate (buffer) diffusion could also be measured. Slower small molecule diffusion is expected when interacting with larger molecules. Sucrose was found to have no interactions with F023700912 consistent with preferential sucrose exclusion resulting in preferential protein hydration. Acetate-protein interactions were clearly detected in samples with no sucrose: acetate-protein interactions were reduced by increasing sucrose concentrations. Increased hydration then results in acetate displacement from the F023700912 molecule. Direct water R2 measurements support an increase in protein hydration as a function of increasing sucrose concentrations.

Sucrose-dependent changes in protein dynamics were measured by R2 relaxation studies in which linker and V_{HH} atoms could easily be distinguished. V_{HH} domain dynamics were slightly elevated from expected from viscosity alone, consistent with increased transient V_{HH}-V_{HH} interactions from compaction. L₁-linker dynamics were profoundly different; samples with higher amounts of sucrose showed faster linker dynamics, clearly trending in an opposite direction expected from measured solution viscosity.

Finally, sucrose stabilized F023700912 was determined to be structurally and conformationally the same as F023700912 with no sucrose. While no direct sucrose-protein binding was observed, changes in other data sets suggest that sucrose is acting indirectly (modulating water-protein, acetate-protein interactions) to affect the dynamic behavior of the L1 linker and folded domains. Sucrose stabilized F023700912 shows increased hydration, greater compaction, increased transient V_{HH} interactions and rapid linker motions when compared with F023700912 formulated without sucrose. Motions associated with aggregation are quenched by hydration. These same motions might also be present in the V_{HH}; they are perhaps amplified in the linker which is not stabilized by protein folding and is completely accessible to solvent. Changes in linker motions were observed for sucrose stabilization of the trivalent V_{HH} based CTLA4 binder; no compaction was detected.

1. A formulation comprising:
(i) about 10 mg/mL to about 200 mg/mL of a trivalent V_{HH} based CTLA4 binder;
(ii) about 5 mM to about 20 mM buffer;
(iii) about 6% to about 16% weight/volume non-reducing sugar;
(iv) about 0.01% to about 0.10% w/v non-ionic surfactant; and
(v) about 1 mM to about 20 mM anti-oxidant.

2. The formulation of claim 1, wherein the trivalent V_{HH} based CTLA4 binder comprises three CDRs comprising SEQ ID NO: 2, SEQ ID NO: 3, and SEQ ID NO: 4 or SEQ ID NO: 5, SEQ ID NO: 6, and SEQ ID NO: 7.

3. The formulation of claim 1, wherein the trivalent V_{HH} based CTLA4 binder comprises amino acid sequence SEQ ID NO: 13 or SEQ ID NO: 15.

4. The formulation of claim 1, wherein the formulation has a pH between 5.0 and 6.0.

5. The formulation of claim 1, wherein the formulation additionally comprises about 6% to about 9% weight/volume (w/v) non-reducing sugar.

6. The formulation of claim 5, wherein the buffer is a sodium acetate buffer or a L-histidine buffer, the non-reducing sugar is sucrose, the non-ionic surfactant is polysorbate 80, and the anti-oxidant is L-methionine.

7. The formulation of claim 6, wherein the buffer is about 8 mM to about 12 mM of sodium acetate buffer.

8. The formulation of claim 6, wherein the sodium acetate buffer is present at a concentration of from about 5 mM to about 10 mM.

9. The formulation of claim 6, wherein the polysorbate 80 is present at a weight ratio of approximately 0.02% w/v.

10. The formulation of claim 1, comprising about 10 mg/mL to about 100 mg/mL of the trivalent V_{HH} based CTLA4 binder.

11. The formulation of claim 10, wherein the concentration of the trivalent V_{HH} based CTLA4 binder is about 10 mg/mL, about 12.5 mg/mL, about 25 mg/mL, about 50 mg/mL, about 75 mg/mL or about 100 mg/mL.

12-15. (canceled)

16. The formulation of claim 1, wherein the formulation has a pH of about 4.8 to about 5.5.

17. The formulation of claim 16, wherein the formulation has a pH of about 5.0.

18. The formulation of claim 1, further comprising a chelator.

19. The formulation of claim 18, wherein the chelator is DTPA.

20. The formulation of claim 1, wherein the formulation is contained in a glass vial or an injection device.

21. The formulation of claim 1, wherein the formulation is a liquid formulation.

22. The formulation of claim 1, wherein after 12 months at 5° C.:
(i) the % monomer of the trivalent V_{HH} based CTLA4 binder is ≥95% Main Peak as determined by size exclusion chromatography;
(ii) the % heavy chain of the trivalent V_{HH} based CTLA4 binder is ≥90% Purity as measured by reduced CE-SDS;
(iii) the % heavy chain of the trivalent V_{HH} based CTLA4 binder is ≥95% as measured reduced CE-SDS;
(iv) the % intact IgG of the trivalent V_{HH} based CTLA4 binder is ≥90% as measured by non-reduced CE-SDS; or
(v) % intact IgG of the trivalent V_{HH} based CTLA4 binder is ≥95% as measured by non-reduced CE-SDS.

23-39. (canceled)

40. A method of treating cancer or chronic infection in a human patient in need thereof, the method comprising administering an effective amount of the formulation of claim 1.

41. (canceled)


SEQUENCE LISTING (22 sequences)

SEQ ID NO: 1 - protein, 119 aa, Lama sp.
DVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSS

SEQ ID NO: 2 - protein, 5 aa, Lama sp.
FYGMG

SEQ ID NO: 3 - protein, 17 aa, Lama sp.
DIRTSAGRTYYADSVKG

SEQ ID NO: 4 - protein, 10 aa, Lama sp.
EPSGISGWDY

SEQ ID NO: 5 - protein, 10 aa, Lama sp.
GGTFSFYGMG

SEQ ID NO: 6 - protein, 10 aa, Lama sp.
DIRTSAGRTY

SEQ ID NO: 7 - protein, 10 aa, Lama sp.
EPSGISGWDY

SEQ ID NO: 8 - protein, 6 aa, Homo sapiens
VRVTVL

SEQ ID NO: 9 - protein, 9 aa, Homo sapiens
ADSQVTEVC

SEQ ID NO: 10 - protein, 14 aa, Homo sapiens
CKVELMYPPPYYLG

SEQ ID NO: 11 - protein, 119 aa, synthetic construct
  REGION 1..119: note = Humanized lama
  VAR_SEQ 1: note = X is D or E
XVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSS

SEQ ID NO: 12 - DNA, 1272 bp, synthetic construct
  misc_feature 1..1272: note = Humanized lama
  source 1..1272: mol_type = other DNA
gacgtgcaattggtggagtctgggggaggagtggtgcagccggggggctctctgagactctcctgtgcagcctctggtggcaccttcagtttctatggcatgggctggttccgccaggctccagggaaggagcgcgagtttgtagcagatattagaaccagtgctggtaggacatactatgcagactccgtgaagggccgattcaccatctccagagacaacagcaagaacacggtgtatctgcaaatgaacagcctgcgccctgaggacacggccctgtattactgtgcagcagagccaagtggaataagtggttgggactactggggccaggggaccctggtcacggtctcctccggaggcggtgggtcaggtggcggaggcagcggtggaggaggtagtggcggtggcggtagtgggggtggaggcagcggaggcggaggcagtgggggcggtggatccgaggtgcagttggtggagtctgggggaggagtggtgcagccggggggctctctgagactctcctgtgcagcctctggtggcaccttcagtttctatggcatgggctggttccgccaggctccagggaaggagcgcgagtttgtagcagatattagaaccagtgctggtaggacatactatgcagactccgtgaagggccgattcaccatctccagagacaacagcaagaacacggtgtatctgcaaatgaacagcctgcgccctgaggacacggccctgtattactgtgcagcagagccaagtggaataagtggttgggactactggggccaggggaccctggtcacggtctcgagcggaggcggtgggtcaggtggcggaggcagcggtggaggaggtagtggcggtggcggtagtgggggtggaggcagcggaggcggaggcagtgggggcggtggctcagaggtacaactagtggagtctggaggtggcgttgtgcaaccgggtaacagtctgcgccttagctgcgcagcgtctggctttaccttcagctcctttggcatgagctgggttcgccaggctccgggaaaaggactggaatgggtttcgtctattagcggcagtggtagcgatacgctctacgcggactccgtgaagggccgtttcaccatctcccgcgataacgccaaaactacactgtatctgcaaatgaatagcctgcgtcctgaagatacggccctgtattactgtactattggtggctcgttaagccgttcttcacagggtaccctggtcaccgtctcctcagcg

SEQ ID NO: 13 - protein, 424 aa, synthetic construct
  REGION 1..424: note = Humanized lama
DVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSSGGGGSGGGGSGGGGSGGGGSGGGGSGGGGSGGGGSEVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 14 - DNA, 810 bp, synthetic construct
  misc_feature 1..810: note = Humanized lama
  source 1..810: mol_type = other DNA
gacgtgcaattggtggagtctgggggaggagtggtgcagccggggggctctctgagactctcctgtgcagcctctggtggcaccttcagtttctatggcatgggctggttccgccaggctccagggaaggagcgcgagtttgtagcagatattagaaccagtgctggtaggacatactatgcagactccgtgaagggccgattcaccatctccagagacaacagcaagaacacggtgtatctgcaaatgaacagcctgcgccctgaggacacggccctgtattactgtgcagcagagccaagtggaataagtggttgggactactggggccaggggaccctggtcacggtctcctccggaggcggtgggtcaggtggcggaggcagcggtggaggaggtagtggcggtggcggtagtgggggtggaggcagcggaggcggaggcagtgggggcggtggatccgaggtgcagttggtggagtctggaggtggcgttgtgcaaccgggtaacagtctgcgccttagctgcgcagcgtctggctttaccttcagctcctttggcatgagctgggttcgccaggctccgggaaaaggactggaatgggtttcgtctattagcggcagtggtagcgatacgctctacgcggactccgtgaagggccgtttcaccatctcccgcgataacgccaaaactacactgtatctgcaaatgaatagcctgcgtcctgaagatacggccctgtattactgtactattggtggctcgttaagccgttcttcacagggtaccctggtcaccgtctcctcagcg

SEQ ID NO: 15 - protein, 270 aa, synthetic construct
  REGION 1..270: note = Humanized lama
DVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 16 - protein, 35 aa, synthetic construct
  REGION 1..35: note = Humanized lama
GGGGSGGGGSGGGGSGGGGSGGGGSGGGGSGGGGS

SEQ ID NO: 17 - protein, 116 aa, synthetic construct
  REGION 1..116: note = Humanized lama
  VAR_SEQ 11: note = X is L or V
  VAR_SEQ 93: note = X is L or V
EVQLVESGGGXVQPGNSLRLSCAASGFTFSSFGMSWVRQAPGKGLEWVSSISGSGSDTLYADSVKGRFTISRDNAKTTLYLQMNSLRPEDTAXYYCTIGGSLSRSSQGTLVTVSSA

SEQ ID NO: 18 - protein, 10 aa, Lama sp.
GFTFSSFGMS

SEQ ID NO: 19 - protein, 17 aa, Lama sp.
SISGSGSDTLYADSVKG

SEQ ID NO: 20 - protein, 6 aa, Lama sp.
GGSLSR

SEQ ID NO: 21 - protein, 85 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 22 - protein, 138 aa, Homo sapiens
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFHHHHHHHHHGGQ